(19) United States
(12) Patent Application Publication
(10) Pub. No.: US 2024/0173453 A1
(43) Pub. Date: May 30, 2024

(54) ULTRAVIOLET LIGHT FLUID TREATMENT DEVICE

(71) Applicant: NICHIA CORPORATION, Anan-shi (JP)

(72) Inventors: Masahiro IMAI, Chino-shi (JP); Naoki NAKAMURA, Suwa-gun (JP)

(73) Assignee: NICHIA CORPORATION, Anan-shi (JP)

(21) Appl. No.: 18/520,490

(22) Filed: Nov. 27, 2023

(30) Foreign Application Priority Data
Nov. 29, 2022 (JP) 2022-190639
Apr. 11, 2023 (JP) 2023-063967
Jun. 1, 2023 (JP) 2023-091003

(51) Int. Cl. A61L 9/20; C02F 1/32

(52) U.S. Cl. CPC A61L 9/20; C02F 1/325; A61L 2209/12; C02F 2201/328; C02F 2303/04

(57) ABSTRACT

An ultraviolet light fluid treatment includes a first flow channel in which fluid flows in a first direction; a second flow channel that is connected to a downstream side of the first flow channel and in which the fluid flows in a second direction opposite to the first direction; a first member disposed between the first flow channel and the second flow channel; a light source that emits ultraviolet light to one or both of the first flow channel and the second flow channel; and a first connection portion connecting a downstream end of the first flow channel and an upstream end of the second flow channel. The first member has a first opening connecting the first flow channel and the second flow channel. An area of the first opening is smaller than an area of the first connection portion in a plan view.

### CROSS-REFERENCE TO RELATED APPLICATIONS

This application is based on and claims priority to Japanese Patent Application No. 2022-190639, filed on Nov. 29, 2022, Japanese Patent Application No. 2023-063967, filed on Apr. 11, 2023, and Japanese Patent Application No. 2023-091003, filed on Jun. 1, 2023, the entire contents of which are incorporated herein by reference.

### TECHNICAL FIELD

The disclosure herein relates to an ultraviolet light fluid treatment device.

### BACKGROUND

Japanese Laid-open Patent Publication No. 2018-140001 describes a device that emits ultraviolet light from a light-emitting element into a flow channel in which fluid flows.

### SUMMARY

According to the present disclosure, it is desirable to provide an ultraviolet light fluid treatment device that can enhance treatment effects.

According to an aspect of the present disclosure, an ultraviolet light fluid treatment includes a first flow channel in which fluid flows in a first direction; a second flow channel that is connected to a downstream side of the first flow channel and in which the fluid flows in a second direction opposite to the first direction; a first member disposed between the first flow channel and the second flow channel; a light source configured to emit ultraviolet light to one or both of the first flow channel and the second flow channel; and a first connection portion connecting a downstream end of the first flow channel and an upstream end of the second flow channel. The first member has a first opening connecting the first flow channel and the second flow channel. An area of the first opening is smaller than an area of the first connection portion in a plan view.

### BRIEF DESCRIPTION OF THE DRAWINGS

Other objects and further features of the present invention will be apparent from the following detailed description when read in conjunction with the accompanying drawings, in which:

FIG. 1 is a schematic perspective view of an ultraviolet light fluid treatment device according to a first embodiment;

FIG. 2 is schematic cross-sectional view of the ultraviolet light fluid treatment device taken through II-II of FIG. 1;

FIG. 3A is a schematic perspective view of a light source according to a first example of the first embodiment;

FIG. 3B is a schematic perspective view illustrating a light source according to a second example of the first embodiment;

FIG. 3C is a schematic perspective view illustrating a light source according to a third example of the first embodiment;

FIG. 4 is a schematic plan view of a first member of the ultraviolet light fluid treatment device of FIG. 1;

FIG. 5 is a schematic plan view of a second member of the ultraviolet light fluid treatment device of FIG. 1;

FIG. 6 is a schematic plan view of a third member of the ultraviolet light fluid treatment device of FIG. 1;

FIG. 7 is a schematic plan view of a fourth member of the ultraviolet light fluid treatment device of FIG. 1;

FIG. 8 is a schematic perspective view illustrating an example of an ultraviolet light fluid treatment device according to a second embodiment;

FIG. 9 is a schematic cross-sectional view of the ultraviolet light fluid treatment device taken through IX-IX of FIG. 8;

FIG. 10 is a schematic plan view illustrating an example of a first member of the ultraviolet light fluid treatment device of FIG. 8;

FIG. 11 is an enlarged view of a region L of FIG. 9;

FIG. 12 is an enlarged view of a region M of FIG. 9;

FIG. 13 is a schematic plan view illustrating an example of a second member of the ultraviolet light fluid treatment device of FIG. 8;

FIG. 14 is an enlarged view of a region N of FIG. 9;

FIG. 15 is a schematic plan view illustrating an example of a third member of the ultraviolet light fluid treatment device of FIG. 8;

FIG. 16 is a schematic plan view illustrating an example of a fourth member of the ultraviolet light fluid treatment device of FIG. 8;

FIG. 17 is a schematic plan view illustrating a first member of an ultraviolet light fluid treatment device according to a third embodiment;

FIG. 18 is a schematic plan view illustrating an example of a first member having grooves;

FIG. 19 is a perspective view of jack mechanisms of a light source according to an embodiment;

FIG. 20 is a top view of the jack mechanisms of the light source according to the embodiment;

FIG. 21 is a side view of the jack mechanisms of the light source according to the embodiment;

FIG. 22 is a diagram illustrating a state before the light source is pressed against a first partition wall of a first light source placement portion;

FIG. 23 is a diagram illustrating a state in which the light source is being pressed against the first partition wall of the first light source placement portion;

FIG. 24 is an exploded perspective view illustrating a first example of a method of disposing the jack mechanisms in the ultraviolet light fluid treatment device according to the embodiment; and

FIG. 25 is an exploded perspective view illustrating a second example of a method of disposing the jack mechanisms in the ultraviolet light fluid treatment device according to the embodiment.

### DETAILED DESCRIPTION

In the following, embodiments will be described with reference to the accompanying drawings. In the drawings, the same components are denoted by the same reference numerals. The drawings schematically illustrate the embodiments, and thus scales, intervals, positional relationships, or the like of members are exaggerated, or some of the members may be omitted. An end view illustrating only a cut surface may be used as a cross-sectional view.

In the drawings, in order to indicate directions, an orthogonal coordinate system having an X-axis, a Y-axis, and a Z-axis is used. The X-axis, the Y-axis, and the Z-axis are orthogonal to one another. In the present specification, an axis along a direction normal to a first member of an ultraviolet light fluid treatment device according to an embodiment is the Z-axis. Axes orthogonal to the direction normal to the first member are the X-axis and the Y-axis. In the present specification and the drawings, the expression “in a plan view” refers to viewing an object from the Z-axis direction, that is, from the direction normal to the first member of the ultraviolet light fluid treatment device according to the embodiment. The direction normal to the first member means a direction normal to a surface of the first member facing a first flow channel or a second flow channel of the ultraviolet light fluid treatment device.

### First Embodiment

### Example Overall Configuration

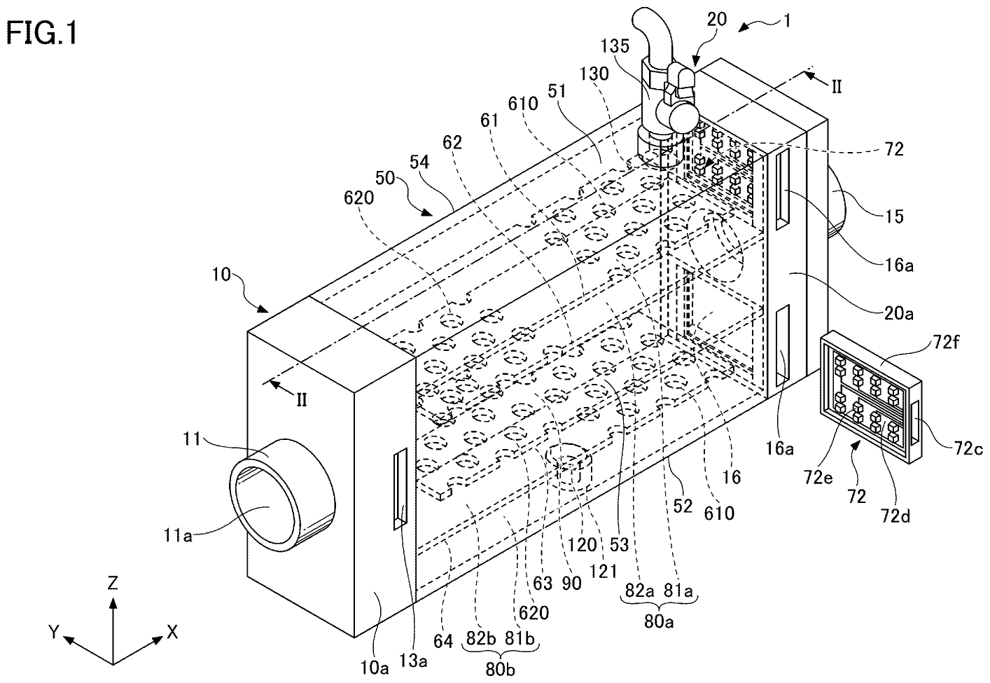

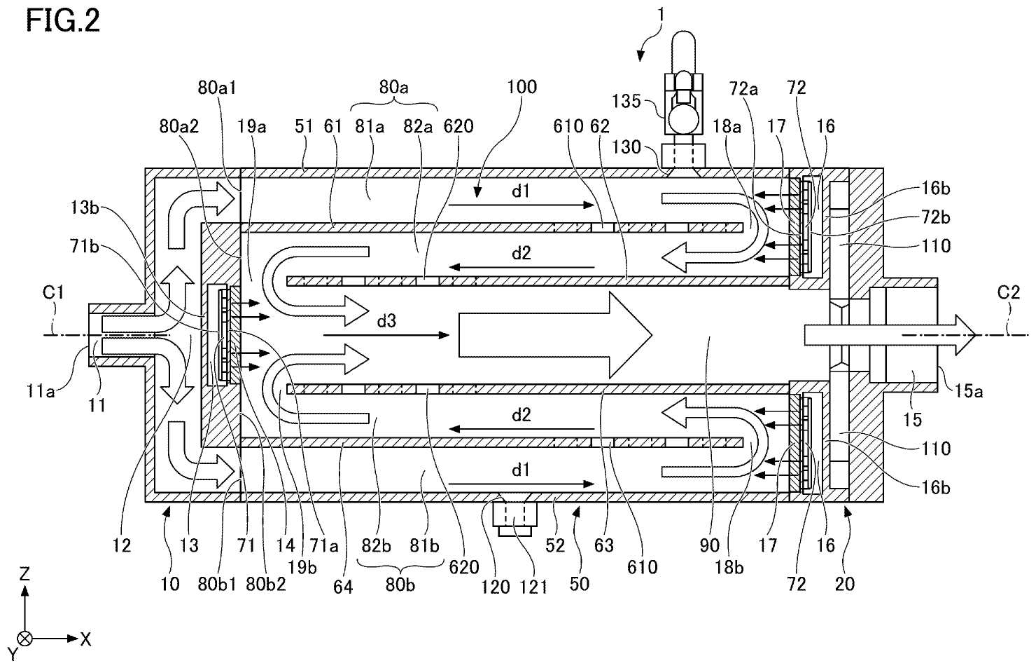

The overall configuration of an ultraviolet light fluid treatment device according to a first embodiment will be described with reference to FIG. 1 and FIG. 2. FIG. 1 is a perspective view of an ultraviolet light fluid treatment device 1 according to the first embodiment. FIG. 2 is schematic cross-sectional view of the ultraviolet light fluid treatment device 1 taken through II-II of FIG. 1. The cross section illustrated in FIG. 2 is parallel to the X-axis and the Z-axis and is orthogonal to the Y-axis.

The ultraviolet light fluid treatment device 1 is configured to treat fluid such as liquid or gas by irradiating the fluid with ultraviolet light. For example, the ultraviolet light fluid treatment device 1 can treat water by irradiating the water flowing in the ultraviolet light fluid treatment device 1 with ultraviolet light so as to reduce the number of bacteria and viruses in the water after the treatment as compared to before the treatment. For example, the ultraviolet light fluid treatment device 1 has a length of 100 mm or more and 1000 mm or less in the X-axis direction, a length of 30 mm or more and 400 mm or less in the Y-axis direction, and a length of 50 mm or more and 800 mm or less in the Z-axis direction. As an example, the ultraviolet light fluid treatment device 1 has a length of 350 mm in the X-axis direction, a length of 100 mm in the Y-axis direction, and a length of 220 mm in the Z-axis direction.

The ultraviolet light fluid treatment device 1 includes a first end portion 10, a second end portion 20, and an intermediate portion 50 between the first end portion 10 and the second end portion 20. Further, the ultraviolet light fluid treatment device 1 includes a first light source 71 and a second light source 72. Further, the ultraviolet light fluid treatment device 1 includes a drain port 120, a drain mechanism 121, an exhaust port 130, and an exhaust mechanism 135. In the example illustrated in FIG. 1 and FIG. 2, the first light source 71 is disposed at the first end portion 10 and the second light source 72 is disposed at the second end portion 20.

The material of the first end portion 10, the second end portion 20, and the intermediate portion 50 is metal, and is, for example, stainless steel. The first end portion 10, the second end portion 20, and the intermediate portion 50 may be members separated from one another, or may be integrated into one another. If there are assumed to be two members, the term “separate members” refers to the two members that contact each other and are not bonded to each other, or the two members that are bonded to each other via an adhesive member or the like.

In FIG. 2, the flows of fluid such as liquid or gas are indicated by white arrows. The fluid flows into the first end portion 10 from the outside of the ultraviolet light fluid treatment device 1. The fluid further flows from the first end portion 10 through the intermediate portion 50 to the second end portion 20, and flows out from the second end portion 20 to the outside of the ultraviolet light fluid treatment device 1.

The first end portion 10 includes an inlet 11 of the fluid, an upstream flow channel 12, a first light source placement portion 13, and a first window 14.

The inlet 11 includes a hole leading from the outside of the ultraviolet light fluid treatment device 1 to the inside of the first end portion 10. An external pipe is connected to the inlet 11, and the fluid flows into the inlet 11 from the external pipe. The cross-sectional shape of the inlet 11 orthogonal to the direction in which the fluid flows is, for example, a circular shape. The inlet 11 includes an inlet port 11a formed as a circular opening, for example. A central axis C1 passing through the center of the circular cross-sectional shape of the inlet 11 is parallel to the X-axis direction.

The upstream flow channel 12 is connected to the inlet 11 within the first end portion 10. The upstream flow channel 12 includes a plurality of branched flow channels. The fluid entering from the inlet 11 into the upstream flow channel 12 flows into the branched flow channels. In the example illustrated in FIG. 2, the upstream flow channel 12 branches into two flow channels from the inlet 11. For example, the upstream flow channel 12 branches from the inlet 11 in directions opposite to each other in the Z-axis direction that is orthogonal to the central axis C1.

The first light source placement portion 13 is formed as a space within the first end portion 10 in which the first light source 71 can be placed. As illustrated in FIG. 1, a first light source opening 13a leading to the first light source placement portion 13 is formed in a lateral surface 10a of the first end portion 10. The first light source 71 can be detachably attached to the first light source placement portion 13 through the first light source opening 13a. The first light source placement portion 13 is a space separated from each flow channel of the ultraviolet light fluid treatment device 1, and the first light source 71 is not exposed to the fluid and is protected from the fluid. For example, if the fluid is liquid, the first light source 71 does not require a waterproof structure. Further, the first light source 71 can be detached, replaced, and maintained while the fluid is flowing in the ultraviolet light fluid treatment device 1. The first light source placement portion 13 may be provided within the intermediate portion 50. In this case, the first light source opening 13a leading to the first light source placement portion 13 is formed in a third wall 53 or a fourth wall 54 of the intermediate portion 50 described later.

The first light source 71 emits ultraviolet light. The peak wavelength of the ultraviolet light emitted from the first light source 71 is, for example, 10 nm or more and 400 nm or less. The first light source 71 includes one or more light-emitting elements. As the light-emitting elements, light emitting diodes (LEDs) or laser diodes (LDs) can be used, for example. As the first light source 71, a light emitting device in which light-emitting elements are mounted on a wiring substrate, a light emitting device in which housings including light-emitting elements are mounted on a wiring substrate, or the like can be used. The first light source 71 has a first surface 71a and a second surface 71b located opposite to the first surface 71a. The first surface 71a is a light emitting surface, and the ultraviolet light is emitted mainly from the first surface 71a.

The first window 14 is disposed to face the first surface 71a of the first light source 71. The first surface 71a is located between the first window 14 and the second surface 71b of the first light source 71 in the X-axis direction, and a portion of the upstream flow channel 12 is located between the second surface 71b and the inlet 11 in the X-axis direction. The first window 14 is formed of a material having transmissivity with respect to the wavelength of the light emitted from the first light source 71. Examples of the material of the first window 14 include inorganic materials formed of at least one material selected from the group consisting of quartz glass, borosilicate glass, calcium fluoride glass, aluminoborosilicate glass, oxynitride glass, chalcogenide glass, and sapphire.

The second end portion 20 includes an outlet 15 of the fluid, a second light source placement portion 16, and a second window 17.

The outlet 15 includes a hole leading from the inside of the second end portion 20 to the outside of the ultraviolet light fluid treatment device 1. An external pipe is connected to the outlet 15. The fluid flowing in the ultraviolet light fluid treatment device 1 flows out from the outlet 15 into the external pipe. The cross-sectional shape of the outlet 15 orthogonal to the direction in which the fluid flows is, for example, a circular shape. The outlet 15 includes an outlet port 15a formed as a circular opening, for example.

A central axis C2 passing through the center of the circular cross-sectional shape of the outlet 15 preferably coincides with the central axis C1 of the inlet 11. Accordingly, the ultraviolet light fluid treatment device 1 can be easily connected to an intermediate portion of a straight pipe that is generally available.

The second light source placement portion 16 is formed as a space within the second end portion 20 in which the second light source 72 can be placed. A plurality of second light sources 72 is disposed within the second end portion 20. For example, two second light sources 72 are disposed within the second end portion 20. In this case, two second light source placement portions 16 are formed within the second end portion 20. The outlet 15 is interposed between the two second light source placement portions 16 in the Z-axis direction.

As illustrated in FIG. 1, a second light source opening 16a leading to a corresponding second light source placement portion 16 is formed in a lateral surface 20a of the second end portion 20. A second light source 72 can be detachably attached to the corresponding second light source placement portion 16 through the second light source opening 16a. The second light source placement portion 16 is a space separated from each flow channel of the ultraviolet light fluid treatment device 1, and the second light source 72 is not exposed to the fluid and is protected from the fluid. For example, if the fluid is liquid, the second light source 72 does not require a waterproof structure. Further, the second light source 72 can be detached, replaced, and maintained while the fluid is flowing in the ultraviolet light fluid treatment device 1. The second light source placement portion 16 may be disposed within the intermediate portion 50. In this case, the second light source opening 16a leading to the second light source placement portion 16 is formed in the third wall 53 or the fourth wall 54 of the intermediate portion 50 described later.

The second light source 72 emits ultraviolet light. As the second light source 72, the same light source as the first light source 71 can be used. As the second light source 72, a light source having an emission peak wavelength different from that of the first light source 71 may be used. The second light source 72 includes a first surface 72a and a second surface 72b located opposite to the first surface 72a. The first surface 72a is a light emitting surface, and the ultraviolet light is emitted mainly from the first surface 72a.

The second window 17 is disposed to face the first surface 72a of the second light source 72. The second window 17 is formed of a material having transmissivity with respect to the wavelength of the light emitted from the second light source 72. Examples of the material of the second window 17 include inorganic materials formed of at least one material selected from the group consisting of quartz glass, borosilicate glass, calcium fluoride glass, aluminoborosilicate glass, oxynitride glass, chalcogenide glass, and sapphire. The material of the second window 17 may be the same as the material of the first window 14. The first surface 72a is located between the second window 17 and the second surface 72b of the second light source 72 in the X-axis direction.

As illustrated in FIG. 1, the second light source 72 includes, for example, a wiring substrate 72d, a plurality of light-emitting elements 72e mounted on the wiring substrate 72d, and a holding member 72f covering the wiring substrate 72d and the light-emitting elements 72e. An insertion port 72c of a connector to be electrically connected to the wiring substrate 72d is formed in the holding member 72f. The above-described first light source 71 can be configured in the same manner as the second light source 72. The first light source 71 and the second light source 72 may both have a waterproof structure. In this case, the first light source placement portion 13 and the second light source placement portion 16 may be provided within a flow channel 100 of the ultraviolet light fluid treatment device 1. Further, light-transmissive members constituting the first window 14 and the second window 17 may be omitted. In addition, the ultraviolet light from the first light source 71 and the second light source 72 may be directly emitted from the first light source placement portion 13 and the second light source placement portion 16 into the flow channel 100.

The second end portion 20 further includes a downstream flow channel 110. The downstream flow channel 110 includes flow channels branching from the outlet 15 in the Z-axis direction. Each of the flow channels faces the second light source placement portion 16. A portion of the fluid, flowing into the outlet 15, can flow into the downstream flow channel 110 and cool the second light source 72 from the second surface 72b side. Accordingly, a decrease in light emission efficiency due to heat generation accompanying light emission of the second light source 72 can be reduced.

Further, the fluid flowing in the upstream flow channel 12 of the first end portion 10 can cool the first light source 71 from the second surface 71b side. Accordingly, a decrease in light emission efficiency due to heat generation accompanying light emission of the first light source 71 can be reduced.

In the example illustrated in FIG. 1, the intermediate portion 50 has four walls (a first wall 51, a second wall 52, the third wall 53, and the fourth wall 54) constituting a housing of the intermediate portion 50. The first wall 51 and the second wall 52 are separated from each other in the Z-axis direction. The third wall 53 and the fourth wall 54 are separated from each other in the Y-axis direction.

Further, the intermediate portion 50 includes a plurality of members 61 to 64 disposed in a space surrounded by the first wall 51, the second wall 52, the third wall 53, and the fourth wall 54. For example, four members (a first member 61, a second member 62, a third member 63, and a fourth member 64) are disposed in the intermediate portion 50.

The first member 61 is disposed between a first flow channel 81a and a second flow channel 82a. The second member 62 is disposed between the second flow channel 82a and a third flow channel 90. The third member 63 is disposed between the third flow channel 90 and a second flow channel 82b. The fourth member 64 is disposed between the second flow channel 82b and a first flow channel 81b.

The first member 61 partitions the first flow channel 81a and the second flow channel 82a. The second member 62 partitions the second flow channel 82a and the third flow channel 90. The third member 63 partitions the third flow channel 90 and the second flow channel 82b. The fourth member 64 partitions the second flow channel 82b and the first flow channel 81b.

Each of the first member 61, the second member 62, the third member 63, and the fourth member 64 is a plate member having a rectangular shape extending in the X-axis direction. In a plan view, each of the first member 61, the second member 62, the third member 63, and the fourth member 64 has a length of 20 mm or more and 500 mm or less in the X-axis direction and a length of 10 mm or more and 200 mm or less in the Y-axis direction. As an example, each of the first member 61, the second member 62, the third member 63, and the fourth member 64 has a length of 183 mm in the X-axis direction and a length of 50 mm in the Y-axis direction. In the present embodiment, the first member 61 has a first opening 610 connecting the first flow channel 81a and the second flow channel 82a. The first opening 610 is a hole penetrating the first member 61 in the Z-axis direction, which corresponds to the thickness direction of the first member 61. The second member 62 has a second opening 620 connecting the second flow channel 82a and the third flow channel 90. The second opening 620 is a hole penetrating the second member 62 in the Z-axis direction, which corresponds to the thickness direction of the second member 62. The third member 63 has a second opening 620 connecting the third flow channel 90 and the second flow channel 82b. The second opening 620 is a hole penetrating the third member 63 in the Z-axis direction, which corresponds to the thickness direction of the third member 63. The fourth member 64 has a first opening 610 connecting the second flow channel 82b and the first flow channel 81b. The first opening 610 is a hole penetrating the fourth member 64 in the Z-axis direction, which corresponds to the thickness direction of the fourth member 64. The first opening 610 of each of the first and fourth members 61 and 64 and the second opening 620 of each of the second and third members 62 and 63 have a diameter of 3 mm or more and 30 mm or less in a plan view. As an example, the first opening 610 and the second opening 620 has a diameter of 10 mm.

The first wall 51, the first member 61, the second member 62, the third member 63, the fourth member 64, and the second wall 52 are separated from one another in the Z-axis direction.

In the Z-axis direction, the first member 61 is disposed between the first wall 51 and the second member 62, the second member 62 is disposed between the first member 61 and the third member 63, the third member 63 is disposed between the second member 62 and the fourth member 64, and the fourth member 64 is disposed between the third member 63 and the second wall 52.

The first member 61, the second member 62, the third member 63, and the fourth member 64 are disposed between the third wall 53 and the fourth wall 54 in the Y-axis direction. Both end portions in the Y-axis direction of each of the first member 61, the second member 62, the third member 63, and the fourth member 64 are supported by the third wall 53 and the fourth wall 54.

One end of the first member 61 is connected to the first end portion 10. The first member 61 extends from a connection portion between the first member 61 and the first end portion 10 toward the second end portion 20. The other end of the first member 61 is separated from the second end portion 20.

One end of the second member 62 is connected to the second end portion 20. The second member 62 extends from a connection portion between the second member 62 and the second end portion 20 toward the first end portion 10. The other end of the second member 62 is separated from the first end portion 10.

One end of the third member 63 is connected to the second end portion 20. The third member 63 extends from a connection portion between the third member 63 and the second end portion 20 toward the first end portion 10. The other end of the third member 63 is separated from the first end portion 10. One end of the fourth member 64 is connected to the first end portion 10.

The fourth member 64 extends from a connection portion between the fourth member 64 and the first end portion 10 toward the second end portion 20. The other end of the fourth member 64 is separated from the second end portion 20. The intermediate portion 50 includes the flow channel 100 connecting the inlet 11 and the outlet 15 and defined by the walls 51 to 54 and the members 61 to 64 described above. The flow channel 100 includes a plurality of branch flow channels 80a and 80b and the third flow channel 90. The branch flow channels 80a and 80b are flow channels branching from the inlet 11. The third flow channel 90 is a flow channel connected to the downstream side of each of the branch flow channels 80a and 80b. The third flow channel 90 is an example of a merged flow channel into which the branch flow channels 80a and 80b merge. In the third flow channel 90, the fluid flows in a third direction d3 opposite to a second direction d2. In the example illustrated in FIG. 1, the third flow channel 90 is located between the branch flow channel 80a and the branch flow channel 80b in the Z-axis direction.

One or both of the branch flow channels 80a and 80b include a first flow channel and a second flow channel. In the example illustrated in FIG. 1, the branch flow channel 80a includes the first flow channel 81a and the second flow channel 82a, and the branch flow channel 80b includes the first flow channel 81b and the second flow channel 82b.

The branch flow channel 80a includes the first flow channel 81a and the second flow channel 82a. The first flow channel 81a is disposed upstream of the second flow channel 82a, and the second flow channel 82a is disposed downstream of the first flow channel 81a. The term “upstream” refers to a side relatively close to the inlet 11 and the term “downstream” refers to a side relatively close to the outlet 15 in each of the flow channels from the inlet 11 toward the outlet 15. In other words, in each of the flow channels in which the fluid flows from the inlet 11 to the outlet 15, the term “upstream” refers to a side to which the fluid flows a relatively short distance from the inlet 11, and the term “downstream” refers to a side from which the fluid flows a relatively short distance to the outlet 15. The branch flow channel 80b includes the first flow channel 81b and the second flow channel 82b. The first flow channel 81b is disposed upstream of the second flow channel 82b, and the second flow channel 82b is disposed downstream of the first flow channel 81b.

The first flow channel 81a of the branch flow channel 80a is defined by the first wall 51, the first member 61, the third wall 53, and the fourth wall 54. The second flow channel 82a of the branch flow channel 80a is defined by the first member 61, the second member 62, the third wall 53, and the fourth wall 54.

The first flow channel 81b of the branch flow channel 80b is defined by the second wall 52, the fourth member 64, the third wall 53, and the fourth wall 54. The second flow channel 82b of the branch flow channel 80b is defined by the third member 63, the fourth member 64, the third wall 53, and the fourth wall 54.

One end of each of the first flow channels 81a and 81b is connected to the upstream flow channel 12 formed within the first end portion 10. The first flow channels 81a and 81b extend in a first direction d1 from respective connection portions connected to the upstream flow channel 12. The first direction d1 is, for example, a direction parallel to the X-axis direction. The fluid flows in each of the first flow channels 81a and 81b in the first direction d1. Further, the first direction d1 may be a direction that is inclined with respect to the X-axis direction.

In the branch flow channel 80a, the first flow channel 81a is connected to the second flow channel 82a through a space between the first member 61 and the second end portion 20. In the branch flow channel 80b, the first flow channel 81b communicates with the second flow channel 82b through a space between the fourth member 64 and the second end portion 20.

The second flow channels 82a and 82b extend in a direction different from the first direction d1 from respective portions communicating with the first flow channels 81a and 81b. The fluid flows in each of the second flow channels 82a and 82b in the second direction d2. The second direction d2 is a direction opposite to the first direction d1.

The first flow channels 81a and 81b, the second flow channels 82a and 82b, and the third flow channel 90 are adjacent to one another in the Z-axis direction. The first flow channel 81a of the branch flow channel 80a is adjacent to the second flow channel 82a of the branch flow channel 80a with the first member 61 interposed therebetween. The first flow channel 81b of the branch flow channel 80b is adjacent to the second flow channel 82b of the branch flow channel 80b with the fourth member 64 interposed therebetween. The third flow channel 90 is adjacent to the second flow channel 82a of the branch flow channel 80a with the second member 62 interposed therebetween, and is adjacent to the second flow channel 82b of the branch flow channel 80b with the third member 63 interposed therebetween. In the Z-axis direction, the two second flow channels 82a and 82b are positioned between the two first flow channels 81a and 81b, and the third flow channel 90 is positioned between the two second flow channels 82a and 82b.

The second flow channel 82a of the branch flow channel 80a is connected to the third flow channel 90 through a space between the second member 62 and the first end portion 10. The second flow channel 82b of the branch flow channel 80b is connected to the third flow channel 90 through a space between the third member 63 and the first end portion 10. The third flow channel 90 extends in the X-axis direction from a portion communicating with the two second flow channels 82a and 82b, and is connected to the outlet 15. The fluid flowing in the second flow channels 82a and 82b merges into the third flow channel 90 and flows in the third flow channel 90 in the third direction d3.

A first connection portion 18a is a portion connecting the downstream end of the first flow channel 81a and the upstream end of the second flow channel 82a. The first connection portion 18a is positioned in the space between the first member 61 and the second end portion 20 in the branch flow channel 80a, for example. A first connection portion 18b is a portion connecting the downstream end of the first flow channel 81b and the upstream end of the second flow channel 82b. The first connection portion 18b is positioned in the space between the fourth member 64 and the second end portion 20 in the branch flow channel 80b, for example.

A second connection portion 19a is a portion connecting the downstream end of the second flow channel 82a and the upstream end of the third flow channel 90. The second connection portion 19a is positioned in the space between the second member 62 and the first end portion 10 in the branch flow channel 80a, for example. A second connection portion 19b is a portion connecting the downstream end of the second flow channel 82b and the upstream end of the third flow channel 90. The second connection portion 19b is positioned in the space between the third member 63 and the first end portion 10 in the branch flow channel 80b, for example.

The first opening 610 is an opening that is formed in a member partitioning a first flow channel and a second flow channel of each of the branch flow channels, and that connects the first flow channel and the second flow channel. The second opening 620 is an opening that is formed in a member partitioning the second flow channel of each of the branch flow channels and a third flow channel, which is the merged flow channel, and that connects the second flow channel and the third flow channel. The number of the branch flow channels (branch flow channel 80a and branch flow channel 80b) is not limited to two, and one branch flow channel or three or more branch flow channels may be provided. In the case of one branch flow channel, a first opening 610 is formed in a member partitioning a first flow channel and a second flow channel, and a second opening 620 is formed in a member partitioning the second flow channel and a third flow channel. In the case of three or more branch flow channels, first openings 610 are formed in members partitioning first flow channels and second flow channels, and second openings 620 are formed in members partitioning the second flow channels and third flow channel(s).

The first light source 71 is disposed at a position where the third flow channel 90 can be irradiated with the ultraviolet light. For example, the first light source 71 is disposed in the first light source placement portion 13 provided in the first end portion 10. The first surface 71a of the first light source 71 faces, through the first window 14, a merging portion of the two second flow channels 82a and 82b into the third flow channel 90. The ultraviolet light emitted from the first surface 71a of the first light source 71 travels from the merging portion of the second flow channels 82a and 82b into the third flow channel 90.

One or more second light sources 72 are disposed at a position where one branch flow channel can be irradiated with the ultraviolet light. The one or more second light sources 72 are disposed at a position where one or both of the first flow channel (81a or 81b) and the second flow channel (82a or 82b) can be irradiated with the ultraviolet light. In the example illustrated in FIG. 2, two second light sources 72 are disposed at respective positions where the branch flow channels 80a and 80b can be irradiated with the ultraviolet light. For example, the two second light sources 72 are disposed in respective second light source placement portions 16 provided in the second end portion 20. The two second light sources 72 are disposed at positions where the first flow channels 81a and 81b and the second flow channels 82a and 82b can be irradiated with the ultraviolet light. In the present embodiment, one second light source 72 of the two second light sources 72 is disposed at a position facing, through the second window 17, a portion where the first flow channel 81a communicates with the second flow channel 82a in the branch flow channel 80a. The other second light source 72 is disposed at a position facing, through the second window 17, a portion where the first flow channel 81b communicates with the second flow channel 82b in the branch flow channel 80b. The ultraviolet light emitted from the first surface 72a of the one second light source 72 travels from the communication portion between the first flow channel 81a and the second flow channel 82a into the first flow channel 81a and the second flow channel 82a. The ultraviolet light emitted from the first surface 72a of the other second light source 72 travels from the communication portion between the first flow channel 81b and the second flow channel 82b into the first flow channel 81b and the second flow channel 82b.

The drain port 120 is a through hole formed in the second wall 52 located on the lower side in the vertical direction of the intermediate portion 50 in the ultraviolet light fluid treatment device 1. The drain port 120 can be opened and closed by a plug. In a state in which the plug of the drain port 120 is opened, liquid inside the ultraviolet light fluid treatment device 1 flows vertically downward by the action of gravity and is discharged through the drain port 120. In a state in which the plug of the drain port 120 is closed, liquid inside the ultraviolet light fluid treatment device 1 is not discharged. The position where the drain port 120 is formed is not particularly limited as long as the drain port 120 is formed in the second wall 52.

The drain mechanism 121 is a mechanism that can adjust the amount of liquid discharged from the drain port 120. For example, the drain mechanism 121 is a drain device including a plug that can adjust opening and closing of the drain port 120. The amount of liquid discharged from the drain port 120 can be adjusted by adjusting opening and closing of the plug of the drain device of the ultraviolet light fluid treatment device 1. The drain mechanism 121 is not an essential component; however, the ultraviolet light fluid treatment device 1 preferably includes the drain mechanism 121 from the viewpoint of improving the workability of a drain operation.

The exhaust port 130 is a through hole formed in the first wall 51 located on the upper side in the vertical direction of the intermediate portion 50 in the ultraviolet light fluid treatment device 1. The exhaust port 130 can be opened and closed by a plug. In a state in which the plug of the exhaust port 130 is opened, air bubbles in the liquid inside the ultraviolet light fluid treatment device 1 flow vertically upward by the action of buoyancy and are discharged through the exhaust port 130. In a state in which the plug of the exhaust port 130 is closed, air bubbles in the liquid inside the ultraviolet light fluid treatment device 1 are not discharged. The position where the exhaust port 130 is formed is not particularly limited as long as the exhaust port 130 is formed in the first wall 51.

The exhaust mechanism 135 is a mechanism that can adjust the amount of air bubbles discharged from the exhaust port 130. For example, the exhaust mechanism 135 is an exhaust device including a plug that can adjust opening and closing of the exhaust port 130. The amount of air bubbles discharged from the exhaust port 130 can be adjusted by adjusting opening and closing of the plug of the ultraviolet light fluid treatment device 1. The exhaust mechanism 135 is not an essential component; however, the ultraviolet light fluid treatment device 1 preferably includes the exhaust mechanism 135 from the viewpoint of improving the workability of an exhaust operation.

For example, if the ultraviolet light fluid treatment device is not used for a certain period of time while the liquid is contained inside the device, mold and bacteria may grow inside the device. Such mold and bacteria would deteriorate treatment effects by the ultraviolet light fluid treatment device. Therefore, in order to prevent the growth of mold and bacteria, the liquid inside the ultraviolet light fluid treatment device is preferably drained periodically or before a period of non-usage, such that no liquid remains in the device, that is, the ultraviolet light fluid treatment device is emptied.

In the present embodiment, the ultraviolet light fluid treatment device 1 includes the drain port 120 on the lower side in the vertical direction. Thus, when the liquid inside the device is discharged to empty the device, the liquid inside the device can be easily discharged through the drain port 120 to the outside. Accordingly, when the ultraviolet light fluid treatment device 1 is emptied, a residual liquid in the device can be reduced. Thus, the growth of mold and bacteria in the device can be reduced, and a deterioration in treatment effects by the ultraviolet light fluid treatment device 1 due to mold and bacteria can be reduced.

Further, if air bubbles are included in the liquid flowing in the flow channels of the ultraviolet light fluid treatment device during treatment, the liquid would not be subjected to the treatment by the volume of the air bubbles, and thus the treatment efficiency of the ultraviolet light fluid treatment device would be decreased. The term “air bubble” refers to gas in the liquid.

In the present embodiment, the ultraviolet light fluid treatment device 1 includes the exhaust port 130 on the upper side in the vertical direction of the intermediate portion 50. Thus, air bubbles in the liquid filled in the device can be reduced. Accordingly, a decrease in the treatment efficiency of the ultraviolet light fluid treatment device 1 can be reduced.

### Example Detailed Configuration of First Light Source 71 and Second Light Sources 72

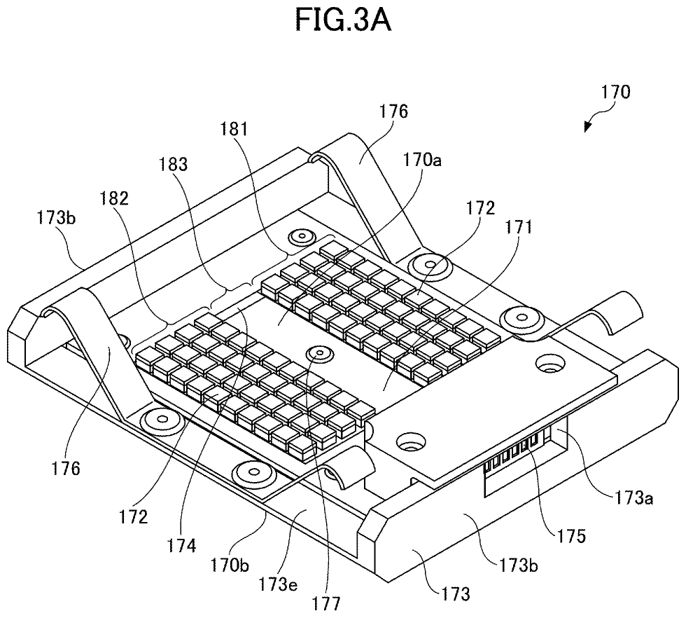

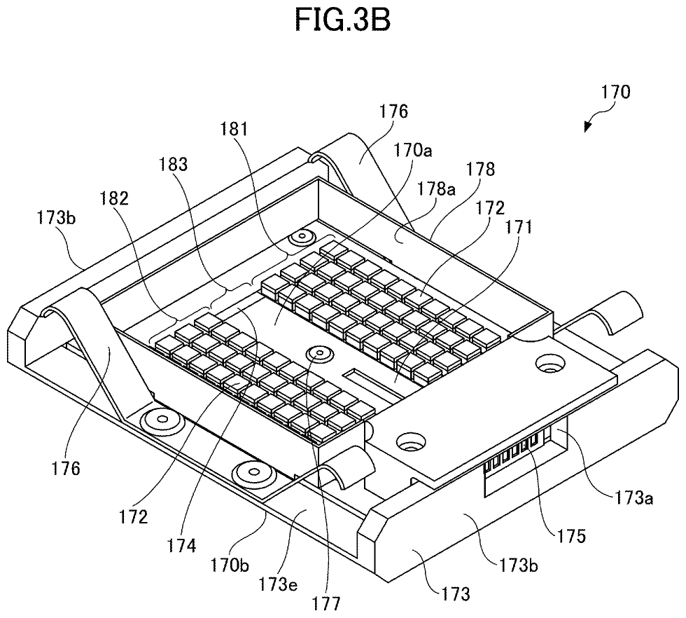

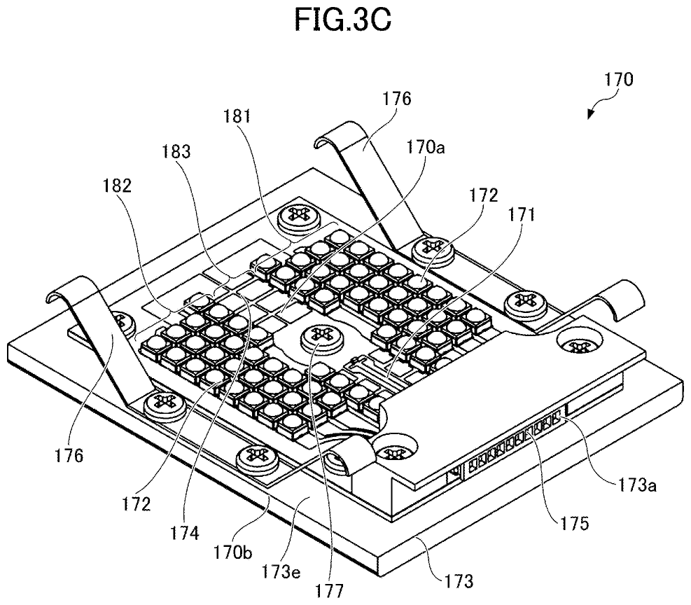

A light source 170 illustrated in FIG. 3A can be used as each of the first light source 71 and the second light sources 72. FIG. 3A is a schematic perspective view illustrating the light source 170 according to a first example. A detailed configuration of the light source 170 will be described while also referring to FIG. 1 and FIG. 2 as appropriate.

As illustrated in FIG. 3A, the light source 170 includes a wiring substrate 171 and a plurality of light-emitting elements. The light-emitting elements are mounted on the surface of the wiring substrate 171. The wiring substrate 171 has, for example, a rectangular shape in a plan view. The center of the wiring substrate 171 is positioned at the intersection of two diagonal lines of the rectangle. The wiring substrate 171 includes a first region 181 and a second region 182. The first region 181 and the second region 182 are arranged side by side in one direction of the wiring substrate 171. The wiring substrate 171 can further include a third region 183. The third region 183 is located between the first region 181 and the second region 182 in a plane parallel to the surface of the wiring substrate 171. The third region 183 includes the center of the wiring substrate 171. If the third region 183 is not provided, the center of the wiring substrate 171 is located, for example, at the boundary between the first region 181 and the second region 182. The first region 181 or the second region 182 may include the center of the wiring substrate 171.

In the example illustrated in FIG. 3A, a plurality of housings 172 is mounted in the first region 181. A plurality of housings 172 is mounted in the second region 182. The housings 172 each includes at least one light-emitting element. Further, each of the housings 172 can include a lens disposed on the at least one light-emitting element. Alternatively, light-emitting elements not housed in the housings 172 may be disposed in the first region 181 and the second region 182. No light-emitting element is disposed in the third region 183.

The width of the third region 183 in the direction in which the first region 181, the third region 183, and the second region 182 are aligned is preferably the same as or greater than the thickness of the first member 61 and the fourth member 64. Accordingly, the amount of light reflected by the first member 61 and the fourth member 64 and returning to the light-emitting element side can be reduced.

The light source 170 can include a holding member 173 that holds the wiring substrate 171. The holding member 173 has a surface 173e on which the wiring substrate 171 is mounted, and has a surface located opposite to the surface 173e. The wiring substrate 171 is fixed to the surface 173e of the holding member 173 by, for example, a screw, an adhesive, or the like. A surface on which the housings 172 including the light-emitting elements are mounted is a first surface 170a of the light source 170, and a surface located opposite to the surface 173e of the holding member 173 is a second surface 170b of the light source 170. The holding member 173 includes a wall portion 173b on the first surface 170a side of the light source 170. The wall portion 173b covers the end portion of the wiring substrate 171. For example, two wall portions 173b are disposed to sandwich the wiring substrate 171 in a plan view.

In the light source 170, wiring 174 electrically connected to the light-emitting elements can be disposed on the surface of the wiring substrate 171. Further, a connector 175 electrically connected to the wiring 174 can be disposed on the surface of the wiring substrate 171. An insertion port 173a exposing the connector 175 from the holding member 173 is disposed in one of the wall portions 173b of the holding member 173.

A spring member 176 is disposed on the first surface 170a side of the light source 170. The spring member 176 is, for example, a metal leaf spring. For example, two spring members 176 sandwich the wiring substrate 171 in a plan view, and are fixed to the holding member 173. The number of the spring members 176 is not limited to two, and may be any number greater than or equal to one. Further, the shape of the spring members 176 is not limited to the shape illustrated in FIG. 3, and the spring members 176 may have any shape.

The light source 170 can used as the first light source and disposed in the first light source placement portion 13 illustrated in FIG. 2. The first surface 170a of the light source 170 disposed in the first light source placement portion 13 faces the first window 14. The ultraviolet light from the first surface 170a is emitted through the first window 14 to the fluid flowing in the third flow channel 90.

The light source 170 is disposed in the first light source placement portion 13 with the spring members 176 being elastically deformed from a natural state. The spring members 176 disposed on the first surface 170a side contact the first window 14. Spacers may be inserted between the first window 14 and the spring members 176, and the spring members 176 may contact the first window 14 via the spacers. The restoring force of the spring members 176 causes the light source 170 to be preloaded toward a first partition wall 13b that partitions the upstream flow channel 12 and the first light source placement portion 13, and the second surface 170b is pressed against the first partition wall 13b. Accordingly, the cooling efficiency of the light source 170 by the fluid flowing in the upstream flow channel 12 can be increased.

Further, the light source 170 can be used as each of the second light sources and disposed in each of the second light source placement portions 16 illustrated in FIG. 2. The first surface 170a of the light source 170 disposed in each of the second light source placement portions 16 faces the second window 17. The ultraviolet light emitted from the first surface 170a is emitted via the second window 17 to the fluid flowing in the branch flow channels 80a and 80b.

The light source 170 is disposed in each of the second light source placement portions 16 with the spring members 176 being elastically deformed from a natural state. The spring members 176 disposed on the first surface 170a side contact the second window 17. Spacers may be inserted between the second window 17 and the spring members 176, and the spring members 176 may contact the second window 17 via the spacers. The restoring force of the spring members 176 causes the light source 170 to be preloaded toward a second partition wall 16b that partitions the downstream flow channel 110 and each of the second light source placement portions 16, and the second surface 170b is pressed against the second partition wall 16b. Accordingly, the cooling efficiency of the light source 170 by the fluid flowing in the downstream flow channel 110 can be increased.

The first region 181 of the light source 170 disposed in the second light source placement portion 16 facing the branch flow channel 80a faces the first flow channel 81a, and light-emitting elements disposed in the first region 181 emit ultraviolet light to the fluid flowing in the first flow channel 81a. The second region 182 of the light source 170 disposed in the second light source placement 16 facing the branch flow channel 80a faces the second flow channel 82a, and light-emitting elements disposed in the second region 182 emit ultraviolet light to the fluid flowing in the second flow channel 82a.

The first region 181 of the light source 170 disposed in the second light source placement portion 16 facing the branch flow channel 80b faces the second flow channel 82b, and light-emitting elements disposed in the first region 181 emit ultraviolet light to the fluid flowing in the second flow channel 82b. The second region 182 of the light source 170 disposed in the second light source placement 16 facing the branch flow channel 80b faces the first flow channel 81b, and light-emitting elements disposed in the second region 182 emit ultraviolet light to the fluid flowing in the first flow channel 81b. The ultraviolet light from the light-emitting elements can be emitted in the extending direction of each of the first flow channels 81a and 81b and the second flow channels 82a and 82b, and thus, the integrated luminance can be increased. The same light-emitting elements can be used as the light-emitting elements disposed in the first region 181 and the light-emitting elements disposed in the second region 182. Light-emitting elements having different emission peak wavelengths may be used as the light-emitting elements disposed in the first region 181 and the light-emitting elements disposed in the second region 182.

The third region 183 of the light source 170 disposed in the second light source placement portion 16 facing the branch flow channel 80a faces the first member 61 through the portion where the first flow channel 81a communicates with the second flow channel 82a in the branch flow channel 80a. No light-emitting element is disposed in the third region 183 facing the first member 61. The third region 183 of the light source 170 disposed in the second light source placement portion 16 facing the branch flow channel 80b faces the fourth member 64 through the portion where the first flow channel 81b communicates with the second flow channel 82b in the branch flow channel 80b. No light-emitting element is disposed in the third region 183 facing the fourth member 64. The integrated luminance of ultraviolet light, emitted from the light-emitting elements disposed in the first region 181 and in the second region 182 to the fluid flowing in the branch flow channels 80a and 80b, can be sufficiently obtained. Therefore, by using a configuration in which no light-emitting element is disposed in the third region 183, the number of light-emitting elements can be reduced while ensuring effects of treating the fluid with the ultraviolet light.

In the light source 170, a screw 177 illustrated in FIG. 3A can be disposed in the third region 183 where no light-emitting element is disposed. The third region 183, including the center of the wiring substrate 171, can be fixed to the holding member 173 by the screw 177. In addition, for example, the four corners of the wiring substrate 171 are fixed to the holding member 173 by screws. By fixing the third region 183 including the center of the wiring substrate 171 to the holding member 173 by the screw 177, a center portion of the wiring substrate 171 can be prevented from being loosened from the holding member 173, and thus, the wiring substrate 171 can be firmly attached to the holding member 173. As a result, the occurrence of a gap between the wiring substrate 171 and the first partition wall 13b can be minimized, and the cooling efficiency of the light source 170 by the fluid flowing through the upstream flow channel 12 can be increased. Further, the occurrence of a gap between the wiring substrate 171 and the second partition wall 16b can be minimized, and the cooling efficiency of the light source 170 by the fluid flowing through the downstream flow channel 110 can be increased.

Further, the light source 170 may include a light-reflecting member. FIG. 3B is a schematic perspective view illustrating a light source 170 including a light-reflecting member 178 according to a second example.

The light-reflecting member 178 has, for example, a shape such as a polygonal shape or a circular shape in a plan view. In the example illustrated in FIG. 3B, the light-reflecting member 178 has a substantially rectangular frame shape in a plan view. Further, the light-reflecting member 178 is a member having a predetermined height from the surface of the wiring substrate 171. The light-reflecting member 178 surrounds the first region 181, the second region 182, and the third region 183 in a plan view.

The light-reflecting member 178 includes for example, a metal material, a resin material, or the like. As the metal material, a material whose surface is treated with aluminum, stainless steel, or the like can be used. As the resin material, a fluororesin or the like can be used.

An inner surface 178a of the light-reflecting member 178 reflects light from the light-emitting elements included in the first region 181 and the second region 182 to the inside of the light-reflecting member 178, such that the amount of light emitted to the outside of the light-reflecting member 178, of the light from the light-emitting elements, can be reduced. Accordingly, the light extraction efficiency of the light source 170 can be improved.

The inner surface 178a of the light-reflecting member 178 is preferably a surface having high reflectivity with respect to the ultraviolet light emitted from the light-emitting elements in order to suppress light loss due to light absorption, light scattering, or the like. Such a surface can be, for example, a surface having a reflectivity of 60% or greater and preferably 90% or greater with respect to the ultraviolet light emitted from the light-emitting elements. Instead of the light-reflecting member 178, a member having light absorbency with respect to the ultraviolet light emitted from the light-emitting elements may be employed.

Further, the arrangement of the housings including the light-emitting elements in the light source 170 is not limited to that illustrated in FIG. 3A and FIG. 3B. FIG. 3C is a schematic perspective view illustrating a light source 170 according to a third example. The arrangement of the housings including the light-emitting elements in the light source 170 illustrated in FIG. 3C differs from that illustrated in FIG. 3A and FIG. 3B.

In addition, as illustrated in FIG. 3C, the holding member 173 of the light source 170 does not necessarily include the wall portions 173b illustrated in FIG. 3A and FIG. 3B, and may be configured with a flat plate without including the wall portions 173b. As described, the arrangement of the housings including the light-emitting elements in the light source 170 is not limited to that illustrated in FIG. 3A and FIG. 3B, and may be changed as appropriate.

<Detailed Example of Fluid Treatment That uses Ultraviolet Light Fluid Treatment Device 1>

Next, an example of fluid treatment that uses the ultraviolet light fluid treatment device 1 according to the present embodiment will be described with continued reference to FIG. 1 through FIG. 3B.

The inlet 11 is connected to the external pipe upstream of the ultraviolet light fluid treatment device 1 directly or via a joint member. The outlet 15 is connected to the external pipe downstream of the ultraviolet light fluid treatment device 1 directly or via a joint member. The fluid flowing through the external pipe upstream of the ultraviolet light fluid treatment device 1 flows into the inlet 11, and is separated into two at the upstream flow channel 12. One portion of the fluid flows into the first flow channel 81a of the branch flow channel 80a, and the other portion of the fluid flows into the first flow channel 81b of the branch flow channel 80b.

The fluid flowing into the first flow channels 81a and 81b flows in each of the first flow channels 81a and 81b in the first direction d1, and then flows into the second flow channel 82a and 82b at the ends, near the second end portion 20, of the first flow channels 81a and 81b. The fluid flowing into the second flow channels 82a and 82b flows in each of the second flow channels 82a and 82b in the second direction d2. The fluid flowing in the first flow channels 81a and 81b and the second flow channels 82a and 82b is irradiated with the ultraviolet light from the second light sources 72.

The fluid flowing in the second flow channels 82a and 82b in the second direction d2 flows into the third flow channel 90. The fluid flowing into the third flow channel 90 flows in the third flow channel 90 in the third direction d3. The fluid flowing in the third flow channel 90 is irradiated with the ultraviolet light from the first light source 71. The fluid flowing in the third flow channel 90 flows out from the outlet 15 to the external pipe connected to the outlet 15.

According to the present embodiment, the fluid flowing into the inside of the ultraviolet light fluid treatment device 1 from the inlet 11 is separated multiple times, merges again, and flows out from the outlet 15. The fluid flowing in the branch flow channels 80a and 80b is irradiated with the ultraviolet light from the second light sources 72. In addition, the fluid flowing from the branch flow channels 80a and 80b into the third flow channel 90 is irradiated with the ultraviolet light from the first light source 71. Accordingly, the integrated luminance of the ultraviolet light emitted to the fluid flowing in the ultraviolet light fluid treatment device 1 can be increased, and effects of treating the fluid with the ultraviolet light can be enhanced.

The first direction d1 in which the fluid flows in the first flow channels 81a and 81b is different from the second direction d2 in which the fluid flows in the second flow channels 82a and 82b. Thus, the lengths of the branch flow channels 80a and 80b can be increased while suppressing an increase in the distance between the inlet 11 and the outlet 15 of the flow channel 100 in the X-axis direction. Further, the first direction d1 and the second direction d2 are opposite to each other. Thus, the lengths of the branch flow channels 80a and 80b can be increased while suppressing an increase in the dimensions of the flow channel 100 in the X-axis direction and the Z-axis direction.

The flow channels of the flow channel 100 preferably overlap one another only in the Z-axis direction in a cross-sectional view. With this configuration, the size of the ultraviolet light fluid treatment device 1 can be easily reduced as compared to a configuration in which flow channels of a flow channel are arranged concentrically one above another. With such a configuration in which flow channels are arranged concentrically one above another, annular members defining the flow channels are separated. Thus, parts management efficiency and assembly efficiency would be decreased. Conversely, in the present embodiment, both end portions of each of the members 61 to 64 in the Y-axis direction are supported by the third wall 53 and the fourth wall 54 constituting part of the housing of the intermediate portion, and thus, the members 61 to 64 can be integrated.

The branch flow channel 80a does not necessarily include the two flow channels (the first flow channel 81a and the second flow channel 82a), and the branch flow channel 80b does not necessarily include the two flow channels (the first flow channel 81b and the second flow channel 82b). Each of the branch flow channels 80a and 80b may include one flow channel or three or more flow channels. As the number of flow channels included in each of the branch flow channels 80a and 80b increases, the lengths of the branch flow channels 80a and 80b increase. Thus, the integrated luminance of the ultraviolet light emitted to the fluid flowing in the branch flow channels 80a and 80b can be increased. As the number of flow channels included in each of the branch flow channels 80a and 80b decreases, pressure loss of the fluid flowing in the ultraviolet light fluid treatment device 1 can be reduced.

If each of the branch flow channels 80a and 80b includes a plurality of flow channels, the fluid flowing in the branch flow channels 80a and 80b can be treated by emitting the ultraviolet light from the second light sources 72 to one or more of the flow channels included in each of the branch flow channels 80a and 80b. By causing the second light sources 72 to emit the ultraviolet light to two or more or all of the flow channels included in each of the branch flow channels 80a and 80b, effects of treating the fluid, flowing in the branch flow channels 80a and 80b, with the ultraviolet light can be enhanced.

If a vortex or turbulence is generated in the flow channel 100, pressure loss of the fluid tends to occur. For this reason, in order to reduce pressure loss of the fluid, a flow velocity difference or a flow rate difference between the fluid flowing from the branch flow channel 80a into the third flow channel 90 and the fluid flowing from the branch flow channel 80b into the third flow channel 90 is preferably reduced.

For example, the cross-sectional shape of each of the first flow channels 81a and 81b in the direction orthogonal to the first direction d1 in which the fluid flows is a rectangular shape. The cross-sectional shape of each of the second flow channels 82a and 82b in the direction orthogonal to the second direction d2 in which the fluid flows is rectangular. The cross-sectional shape of the third flow channel 90 in the direction orthogonal to the third direction d3 in which the fluid flows is a rectangular shape.

Further, the cross-sectional area of the first flow channel 81a in the direction orthogonal to the first direction d1 in which the fluid flows, the cross-sectional area of the first flow channel 81b in the direction orthogonal to the first direction d1 in which the fluid flows, the cross-sectional area of the second flow channel 82a in the direction orthogonal to the second direction d2 in which the fluid flows, and the cross-sectional area of the second flow channel 82b in the direction orthogonal to the second direction d2 in which the fluid flows are the same. Therefore, the cross-sectional area of the branch flow channel 80a in the direction orthogonal to the directions in which the fluid flows is the same as the cross-sectional area of the branch flow channel 80b in the direction orthogonal to the directions in which the fluid flows.

Further, the length of an upstream end 80al of the branch flow channel 80a and a downstream end 80a2 of the branch flow channel 80a is the same as the length of an upstream end 80b1 of the branch flow channel 80b and a downstream end 80b2 of the branch flow channel 80b.

Therefore, according to the present embodiment, a flow velocity difference or a flow rate difference between the fluid flowing from the branch flow channel 80a into the third flow channel 90 and the fluid flowing from the branch flow channel 80b into the third flow channel 90 can be reduced. Accordingly, pressure loss of the fluid due to a vortex or turbulence generated in the flow channel 100 by a difference in the flow rate or the flow velocity of the fluid can be reduced.

The cross-sectional area of the first flow channel 81a of the branch flow channel 80a in the direction orthogonal to the direction in which the fluid flows is greater than or equal to the cross-sectional area of the inlet 11 in the direction orthogonal to the direction in which the fluid flows. For example, the cross-sectional area of the first flow channel 81a in the direction orthogonal to the direction in which the fluid flows is the same as the cross-sectional area of the inlet 11 in the direction orthogonal to the direction in which the fluid flows. The cross-sectional area of the first flow channel 81b of the branch flow channel 80b in the direction orthogonal to the direction in which the fluid flows is greater than or equal to the cross-sectional area of the inlet 11 in the direction orthogonal to the direction in which the fluid flows. For example, the cross-sectional area of the first flow channel 81b in the direction orthogonal to the direction in which the fluid flows is the same as the cross-sectional area of the inlet 11 in the direction orthogonal to the direction in which the fluid flows. Accordingly, the flow velocity of the fluid separated from the inlet 11 and flowing into the first flow channels 81a and 81b can be set to one-half or less of the flow velocity of the fluid flowing in the inlet 11. By reducing the flow velocity of the fluid flowing in the first flow channels 81a and 81b, the integrated illuminance of the ultraviolet light emitted from the second light sources 72 to the fluid flowing in the first flow channels 81a and 81b can be increased, and effects of treating the fluid, flowing in the first flow channels 81a and 81b, with the ultraviolet light can be enhanced.

Further, the cross-sectional area of the second flow channel 82a of the branch flow channel 80a in the direction orthogonal to the direction in which the fluid flows is greater than or equal to the cross-sectional area of the inlet 11 in the direction orthogonal to the direction in which the fluid flows. For example, the cross-sectional area of the second flow channel 82a in the direction orthogonal to the direction in which the fluid flows is the same as the cross-sectional area of the inlet 11 in the direction orthogonal to the direction in which the fluid flows. The cross-sectional area of the second flow channel 82b of the branch flow channel 80b in the direction orthogonal to the direction in which the fluid flows is greater than or equal to the cross-sectional area of the inlet 11 in the direction orthogonal to the direction in which the fluid flows. For example, the cross-sectional area of the second flow channel 82b in the direction orthogonal to the direction in which the fluid flows is the same as the cross-sectional area of the inlet 11 in the direction orthogonal to the direction in which the fluid flows. Accordingly, the flow velocity of the fluid separated from the inlet 11, flowing into the first flow channels 81a and 81b, and then flowing in the second flow channels 82a and 82b can be set to one-half or less of the flow velocity of the fluid flowing in the inlet 11. By reducing the flow velocity of the fluid flowing in the second flow channels 82a and 82b, the integrated illuminance of the ultraviolet light emitted from the second light sources 72 to the fluid flowing in the second flow channels 82a and 82b can be increased, and effects of treating the fluid, flowing in the second flow channels 82a and 82b, with the ultraviolet light can be enhanced.

The fluid from each of the second flow channels 82a and 82b of the two branch flow channels 80a and 80b merges and flows into the third flow channel 90. Therefore, in order to reduce the flow velocity of the fluid flowing in the third flow channel 90, the cross-sectional area of the third flow channel 90 in the direction orthogonal to the direction in which the fluid flows is preferably set to be greater than the cross-sectional areas of the second flow channels 82a and 82b in the direction orthogonal to the direction in which the fluid flows. Accordingly, the integrated illuminance of the ultraviolet light emitted from the first light source 71 to the fluid flowing in the third flow channel 90 can be increased, and effects of treating the fluid, flowing in the third flow channel 90, with the ultraviolet light can be enhanced.

For example, the cross-sectional area of the third flow channel 90 in the direction orthogonal to the direction in which the fluid flows is equal to a sum of the cross-sectional area of the second flow channel 82a in the direction orthogonal to the direction in which the fluid flows and the cross-sectional area of the second flow channel 82b in the direction orthogonal to the direction in which the fluid flows. Therefore, the flow velocity of the fluid flowing in each of the second flow channels 82a and 82b can be approximately the same as the flow velocity of the fluid flowing in the third flow channel 90. By achieving uniform flow velocities or reducing a change in flow velocities, pressure loss of the fluid due to a vortex or turbulence can be reduced.

The third flow channel 90 is not necessarily disposed between the branch flow channels 80a and 80b. For example, the two branch flow channels 80a and 80b illustrated in FIG. 2 may be disposed on the first wall 51 side (in FIG. 2, on the upper side) of the third flow channel 90 in the Z-axis direction, or may be disposed on the second wall 52 side (in FIG. 2, on the lower side) of the third flow channel 90 in the Z-axis direction. For example, the two branch flow channels 80a and 80b disposed on the first wall 51 side or the second wall 52 side of the third flow channel 90 may be adjacent to each other in the Y-axis direction.

### <Detailed Configurations of Members 61 to 64 and Main Effects Thereof>

### (First Member 61)

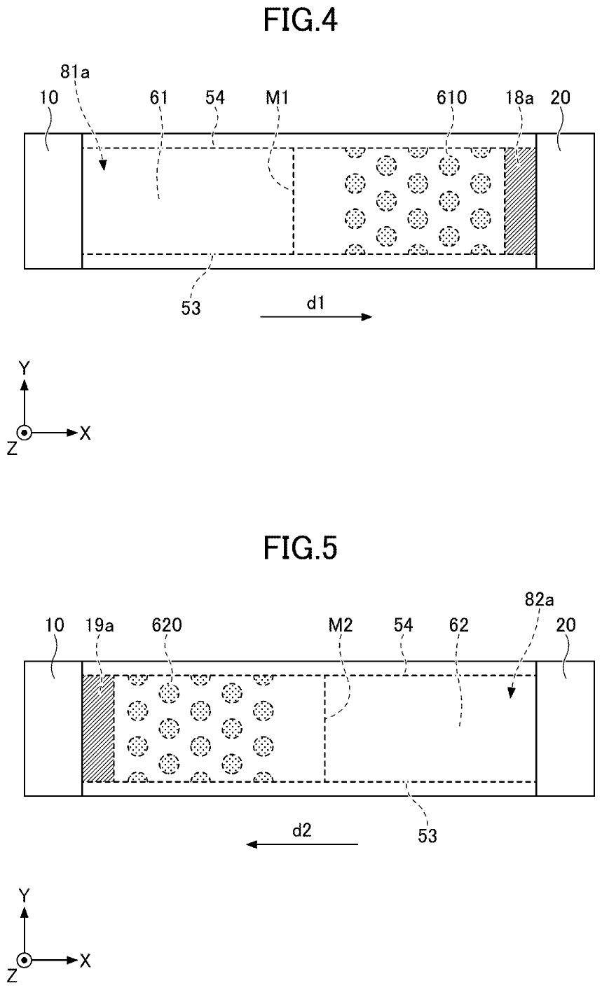

FIG. 4 is a schematic plan view illustrating an example of the first member 61 of the ultraviolet light fluid treatment device 1. FIG. 4 depicts a perspective view of the first member 61 and the first connection portion 18a located inside the ultraviolet light fluid treatment device 1 in a plan view.

In the example illustrated in FIG. 0.4, the first member 61 has a plurality of first openings 610. In the present embodiment, the first openings 610 are provided only in a region downstream of a center M1 of the first member 61 in the first direction d1. No first opening 610 is provided upstream of the center M1 of the first member 61 in the first direction d1.

When the first member 61 is viewed from the first flow channel 81a side in a plan view, the area of each of the first openings 610 is smaller than the area of the first connection portion 18a. The area of one region indicated by dot hatching in FIG. 4 corresponds to the area of one first opening 610 in a plan view. The area of a region indicated by diagonal hatching in FIG. 4 corresponds to the area of the first connection portion 18a in a plan view. When the first member 61 is viewed from the first flow channel 81a side in a plan view, the area of one first opening 610 is smaller than the area of the first connection portion 18a.

In the example illustrated in FIG. 4, the plurality of first openings 610 includes openings having a substantially circular shape in a plan view and openings having a substantially semicircular shape in a plan view. Further, in a plan view, the first openings 610 are arranged in a staggered pattern. The staggered pattern means that objects are not aligned in rows or columns, but are alternately shifted up, down, left, and right.

The number of the first openings 610 is not particularly limited. The first member 61 may have at least one first opening 610. Further, the arrangement of the at least one first opening 610 in the first member 61, the interval between adjacent first openings 610, and the like can be appropriately changed according to the use and the like of the ultraviolet light fluid treatment device 1. The shapes of the first openings 610 in a plan view are not limited to the substantially circular shape and the substantially semicircular shape. The first openings 610 may have a substantially rectangular shape, a substantially elliptical shape, a substantially polygonal shape, or the like in a plan view. The substantially rectangular shape includes a substantially rectangular shape with the longer side being in the direction orthogonal to the first direction d1 in a plan view. Further, the plurality of first openings 610 may include first openings 610 having different shapes in a plan view.

In the present embodiment, the first member 61 has the first openings 610. Therefore, in a case where air bubbles are present in the second flow channel 82a, which is located under the first flow channel 81a with the first member 61 being interposed therebetween, the air bubbles move into the first flow channel 81a through the first openings 610, and then, the air bubbles are easily discharged from the exhaust port 130 to the outside of the device. Accordingly, the accumulation of air bubbles in the first flow channel 81a and the second flow channel 82a can be reduced. Thus, a decrease in treatment efficiency due to air bubbles can be reduced, and treatment effects can be enhanced.

Further, in the present embodiment, the first member 61 has the first openings 610. Therefore, the fluid can be easily discharged through the first openings 610 when the ultraviolet light fluid treatment device is emptied periodically or before a period of non-usage in order to prevent the growth of mold and bacteria. Accordingly, the growth of mold and bacteria in the ultraviolet light fluid treatment device due to residual fluid in the device can be reduced. Thus, a decrease in treatment efficiency due to mold and bacteria can be reduced, and treatment effects by the ultraviolet light fluid treatment device 1 can be improved.

Further, in the present embodiment, the first member 61 has the first openings 610. Therefore, a portion of the fluid flowing in, for example, the first flow channel 81a flows into the second flow channel 82a through the first openings 610. As a result, the flow of the fluid in the second flow channel 82a can be smoothed, and a deviation in the velocity of the fluid flowing in the flow channel can be reduced as compared to when the first member 61 does not have the first openings 610. Accordingly, the time during which the fluid is irradiated with the ultraviolet light increases, and thus, the integrated illuminance can be increased, and treatment effects by the ultraviolet light fluid treatment device 1 can be enhanced.

Further, in the present embodiment, when the first member 61 is viewed from the first flow channel 81a side in a plan view, the area of each of the first openings 610 is smaller than the area of the first connection portion 18a. Therefore, the fluid flows from the first flow channel 81a into the second flow channel 82a mainly through the first connection portion 18a, and thus, the flow of the fluid is less likely to be disturbed by the presence of the first openings 610. Accordingly, treatment effects as described above can be enhanced without disturbing the flow of the fluid.

Further, for example, if the first openings 610 are provided upstream of the center M1 of the first member 61 in the first direction d1, the amount of fluid flowing from the first flow channel 81a to the second flow channel 82a through the first openings 610 would increase. Since the distance in which the fluid flows from the first flow channel 81a to the second flow channel 82a through the first openings 610 located upstream of the center M1 is short, the time during which the fluid is irradiated with the ultraviolet light from the light source 170 would be reduced. As a result, the integrated illuminance of the ultraviolet light emitted to the fluid would decrease, and thus treatment effects would decrease. Conversely, in the present embodiment, since the first openings 610 are provided only in the region downstream of the center M1 of the first member 61 in the first direction d1, a decrease in the distance in which the fluid flows in the first flow channel 81a can be reduced. Thus, a decrease in the integrated luminance can be reduced, and treatment effects by the ultraviolet light fluid treatment device 1 can be enhanced.

Further, in the present embodiment, the ultraviolet light fluid treatment device 1 includes the plurality of branch flow channels 80a and 80b. Therefore, the ultraviolet light fluid treatment device 1 capable of treating a large amount of fluid and improving fluid treatment effects in each of the branch flow channels 80a and 80b can be provided.

### (Second Member 62)

FIG. 5 is a schematic plan view illustrating an example of the second member 62 of the ultraviolet light fluid treatment device 1 of FIG. 1. FIG. 5 depicts a perspective view of the second member 62 and the second connection portion 19a located inside the ultraviolet light fluid treatment device 1 in a plan view.

In the example illustrated in FIG. 5, the second member 62 has a plurality of second openings 620. In the present embodiment, the second openings 620 are provided only in a region downstream of a center M2 of the second member 62 in the second direction d2. No second opening 620 is provided upstream of the center M2 of the second member 62 in the second direction d2.

When the second member 62 is viewed from the second flow channel 82a side in a plan view, the area of each of the second openings 620 is smaller than the area of the second connection portion 19a. The area of one region indicated by dot hatching in FIG. 5 corresponds to the area of one second opening 62 in a plan view. The area of a region indicated by diagonal hatching in FIG. 5 corresponds to the area of the second connection portion 19a in a plan view. When the first member 61 is viewed from the first flow channel 81a side in a plan view, the area of one second opening 62 is smaller than the area of the second connection portion 19a.

Further, in the present embodiment, when the first member 61 is viewed from the first flow channel 81a side in a plan view, the second openings 620 are provided at positions that do not overlap the first openings 610. Specifically, as illustrate in FIG. 4, the first openings 610 are provided downstream of the center M1 of the first member 61 in the first direction d1. Conversely, as illustrate in FIG. 5, the second openings 620 are provided downstream of the center M2 of the second member 62 in the second direction d2. Since the second openings 620 are located opposite to the first openings 610 in the X-axis direction, the second openings 620 do not overlap the first openings 610 when the first member 61 is viewed from the first flow channel 81a side in a plan view.

In the example illustrated in FIG. 5, the plurality of second openings 620 includes openings having a substantially circular shape in a plan view and openings having a substantially semicircular shape in a plan view. Further, in a plan view, the second openings 620 are arranged in a staggered pattern.

The number of the second openings 620 is not particularly limited. The second member 62 may have at least one second opening 620. Further, the arrangement of the at least one second opening 620 in the second member 62, the interval between adjacent second members 62, and the like can be appropriately changed according to the use and the like of the ultraviolet light fluid treatment device 1. The shapes of the second openings 620 in a plan view are not limited to the substantially circular shape and the substantially semicircular shape. The second openings 620 may have a substantially rectangular shape, a substantially elliptical shape, a substantially polygonal shape, or the like in a plan view. The substantially rectangular shape includes a substantially rectangular shape with the longer side being in the direction orthogonal to the second direction d2 in a plan view. Further, the plurality of second openings 620 may include second openings 620 having different shapes in a plan view.

In the present embodiment, the second member 62 has the second openings 620. Therefore, in a case where air bubbles are present in the third flow channel 90, which is located under the second flow channel 82a with the second member 62 being interposed therebetween, the air bubbles move into the second flow channel 82a through the second openings 620. Further, after moving from the second flow channel 82a into the first flow channel 81a through the first openings 610, the air bubbles can be easily discharged from the exhaust port 130 to the outside of the ultraviolet light fluid treatment device. Accordingly, the accumulation of air bubbles in the second flow channel 82a and the third flow channel 90 can be reduced. Thus, a decrease in treatment efficiency due to air bubbles can be reduced, and treatment effects by the ultraviolet light fluid treatment device 1 can be enhanced.

Further, in the present embodiment, the second member 62 has the second openings 620. Therefore, the fluid can be easily discharged through the second openings 620 when the ultraviolet light fluid treatment device is emptied in order to prevent the growth of mold and bacteria. Accordingly, the growth of mold and bacteria in the ultraviolet light fluid treatment device due to residual fluid in the ultraviolet light fluid treatment device can be reduced. Thus, a decrease in treatment efficiency due to mold and bacteria can be reduced, and treatment effects by the ultraviolet light fluid treatment device 1 can be enhanced.

Further, in the present embodiment, the second member 62 has the second openings 620. Therefore, a portion of the fluid flowing in, for example, the second flow channel 82a flows into the third flow channel 90 through the second openings 620. As a result, the flow of the fluid in the third flow channel 90 can be smoothed, and a deviation in the velocity of the fluid flowing in the flow channel can be reduced as compared to when the second member 62 does not have the second openings 620. Accordingly, treatment effects by the ultraviolet light fluid treatment device 1 can be enhanced.

Further, in the present embodiment, when the second member 62 is viewed from the second flow channel 82a side in a plan view, the area of each of the second openings 620 is smaller than the area of the second connection portion 19a. Therefore, the fluid flows from the second flow channel 82a into the third flow channel 90 mainly through the second connection portion 19a, and thus, the flow of the fluid is less likely to be disturbed by the presence of the second openings 620. Accordingly, treatment effects as described above can be enhanced without disturbing the flow of the fluid.

Further, for example, if the second openings 620 are located upstream of the center M2 of the second member 62 in the second direction d2, the amount of fluid flowing from the second flow channel 82a to the third flow channel 90 through the second openings 620 would increase. Since the distance in which the fluid flows from the second flow channel 82a to the third flow channel 90 through the second openings 620 located upstream of the center M2 is short, the time during which the fluid is irradiated with the ultraviolet light from the light source 170 would be reduced. As a result, the integrated illuminance of the ultraviolet light emitted to the fluid would decrease, and thus the treatment effects would decrease. Conversely, in the present embodiment, since the second openings 620 are provided only in the region downstream of the center M2 of the second member 62 in the second direction d2, a decrease in the distance in which the fluid flows in the second flow channel 82a can be reduced. Thus, a decrease in the integrated luminance can be reduced, and the treatment effects by the ultraviolet light fluid treatment device 1 can be enhanced.

Further, in the present embodiment, when the first member 61 is viewed from the first flow channel 81a side in a plan view, the second openings 620 are provided at positions that do not overlap the first openings 610. Therefore, the second openings 620 can be spaced apart from the first openings 610 in a plan view. Accordingly, as compared to when the ultraviolet light is emitted to the fluid flowing in the shortest path from the first openings 610 to the second openings 620, the integrated illuminance of the ultraviolet light emitted to the fluid can be increased. Thus, treatment effects by the ultraviolet light fluid treatment device 1 can be enhanced.

### (Third Member 63 and Fourth Member 64)

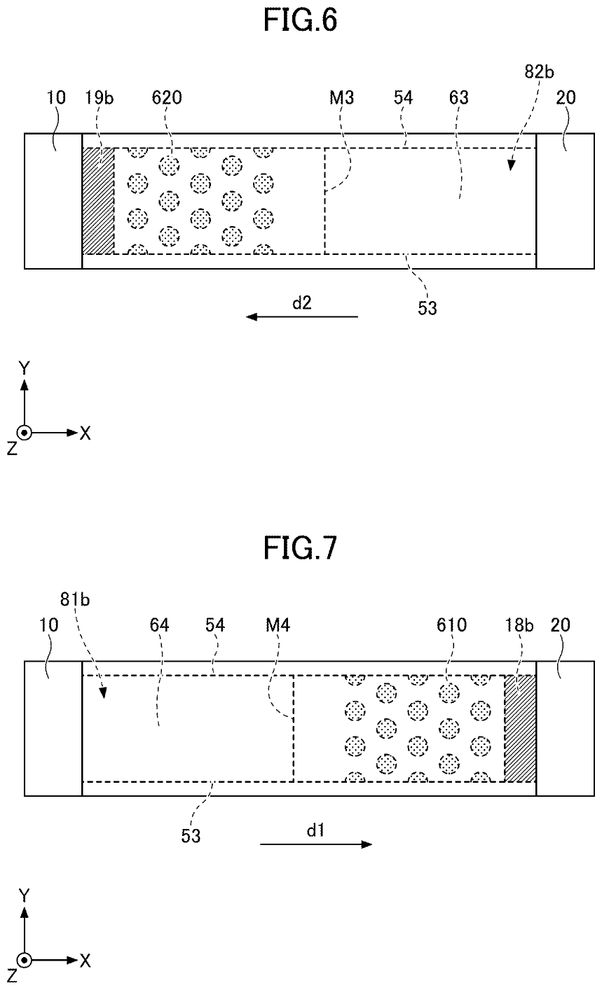

FIG. 6 is a schematic plan view illustrating an example of the third member 63 of the ultraviolet light fluid treatment device 1. FIG. 6 depicts a perspective view of the third member 63 and the second connection portion 19b located inside the ultraviolet light fluid treatment device 1 in a plan view. FIG. 7 is a schematic plan view illustrating an example of the fourth member 64 of the ultraviolet light fluid treatment device 1. FIG. 7 depicts a perspective view of the fourth member 64 and the first connection portion 18b located inside the ultraviolet light fluid treatment device 1 in a plan view.

The configuration of the third member 63 is substantially the same as the configuration of the second member 62, except that the area of each of the second openings 620 is smaller than the area of the second connection portion 19b when the third member 63 is viewed from the second flow channel 82b side in a plan view. Effects of the third member 63 are substantially the same as those of the second member 62. The configuration of the fourth member 64 is substantially the same as the configuration of the first member 61, except that the area of each of the first openings 610 is smaller than the area of the first connection portion 18b when the fourth member 64 is viewed from the first flow channel 81b side in a plan view. Effects of the fourth member 64 are substantially the same as those of the first member 61. Therefore, the description of the third member 63 and the fourth member 64 the same as those described above will not be repeated.

### Second Embodiment

Next, an ultraviolet light fluid treatment device according to a second embodiment will be described. Members having the same names and reference numerals as those of the first embodiment and modifications thereof denote the same or similar members, and a detailed description thereof will be omitted as appropriate.

The second embodiment mainly differs from the first embodiment in that a first member includes a first portion, a third opening, and a third portion, and a second member includes a second portion.

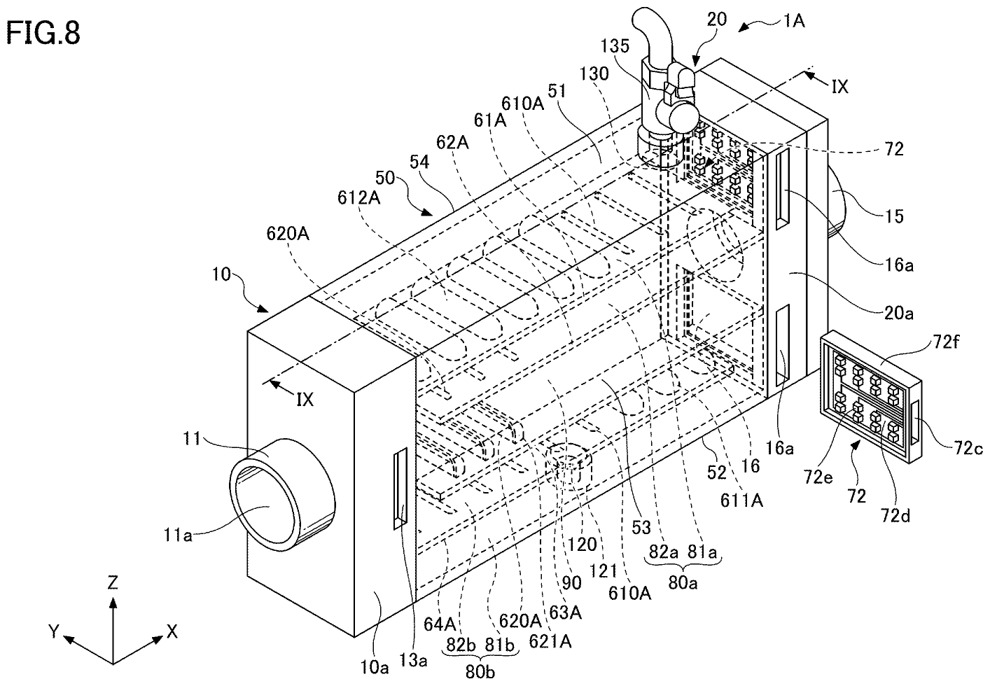

FIG. 8 is a schematic perspective view illustrating an example of an ultraviolet light fluid treatment device 1A according to the second embodiment.

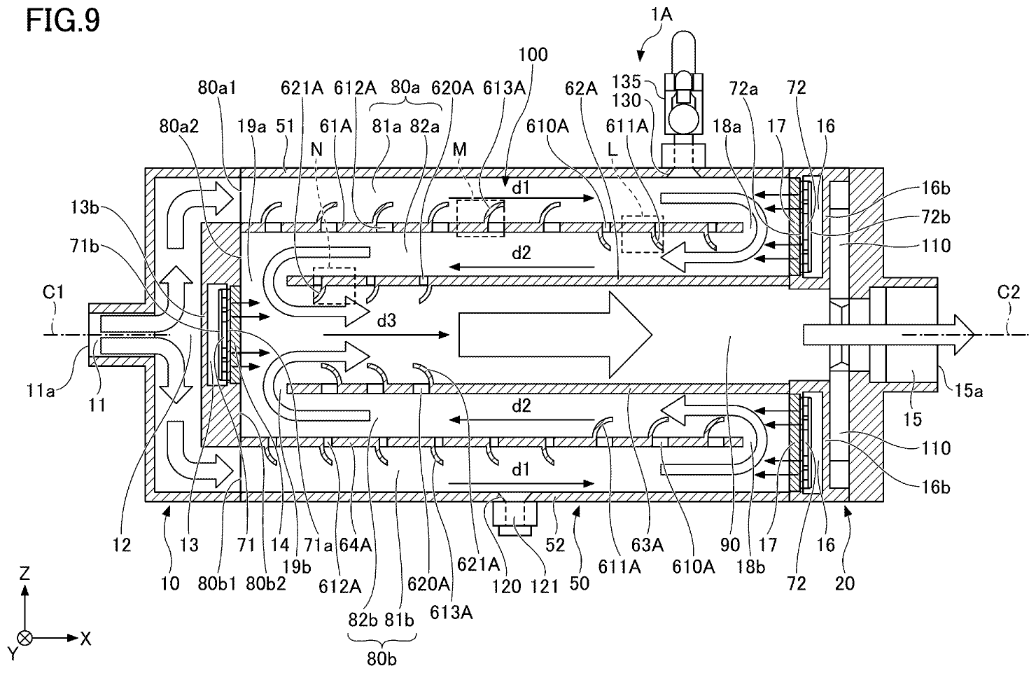

FIG. 9 is a schematic cross-sectional view of the ultraviolet light fluid treatment device 1A taken through IX-IX of FIG. 8.

As illustrated in FIG. 8 and FIG. 9, the ultraviolet light fluid treatment device 1A includes a first member 61A, a second member 62A, a third member 63A, and a fourth member 64A.

The first member 61A has a first opening 610A connecting the first flow channel 81a and the second flow channel 82a. Further, the first member 61A includes a first portion 611A that extends from a region adjacent to the first opening 610A into the second flow channel 82a. In addition, the first member 61A has a third opening 612A located upstream of the first opening 610A of the first member 61A in the first direction d1 and connecting the first flow channel 81a and the second flow channel 82a. Further, the first member 61A includes a third portion 613A that extends from a region adjacent to the third opening 612A into the first flow channel 81a. Each of the first opening 610A and the third opening 612A is a hole penetrating the first member 61A in the thickness direction of the first member 61A.

The second member 62A has a second opening 620A connecting the second flow channel 82a and the third flow channel 90. Further, the second member 62A includes a second portion 621A that extends from a region adjacent to the second opening 620A into the third flow channel 90. The second opening 620A is a hole penetrating the second member 62A in the thickness direction of the second member 62A.

The third member 63A has a second opening 620A connecting the second flow channel 82b and the third flow channel 90. Further, the third member 63A includes a second portion 621A that extends from a region adjacent to the second opening 620A into the third flow channel 90. The second opening 620A is a hole penetrating the third member 63A in the thickness direction of the third member 63A.

The fourth member 64A has a first opening 610A connecting the first flow channel 81b and the second flow channel 82b. Further, the fourth member 64A includes a first portion 611A that extends from a region adjacent to the first opening 610A into the second flow channel 82b. In addition, the fourth member 64A has a third opening 612A located upstream of the first opening 610A of the fourth member 64A in the first direction d1 and connecting the first flow channel 81b and the second flow channel 82b. Further, the fourth member 64A includes a third portion 613A that extends from a region adjacent to the third opening 612A into the first flow channel 81b. Each of the first opening 610A and the third opening 612A is a hole penetrating the fourth member 64A in the thickness direction of the fourth member 64A.

### <Detailed Configurations of Members 61A to 64A and Main Effects Thereof>

### (First Member 61A)

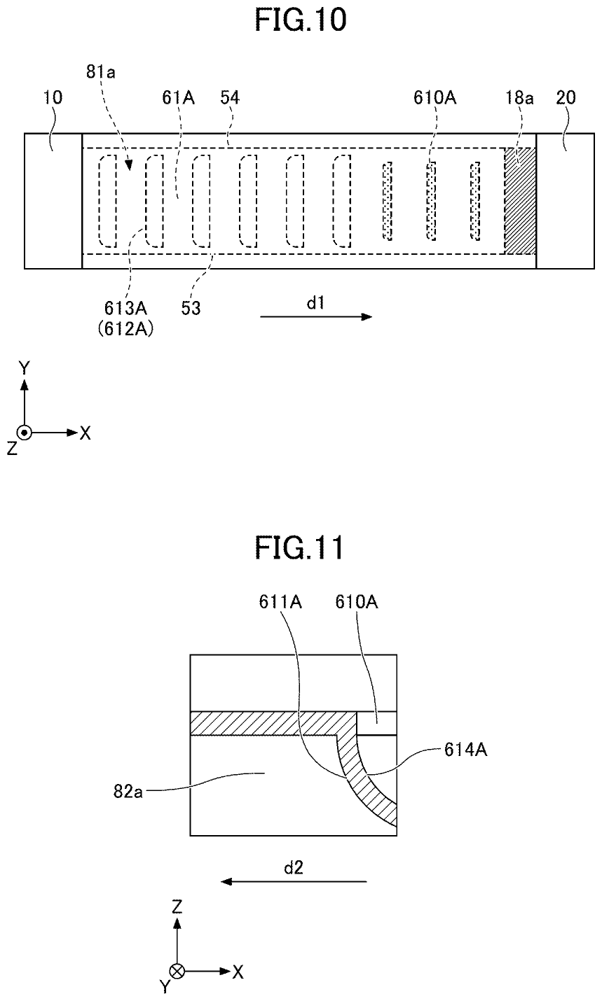

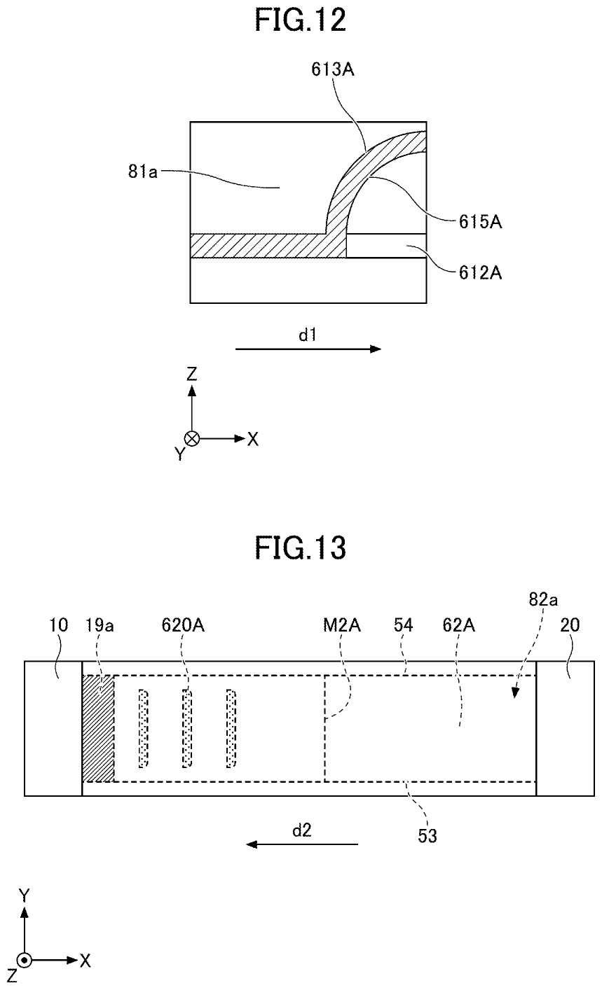

A detailed configuration of the first member 61A will be described with reference to FIG. 10 through FIG. 12. FIG. 10 is a schematic plan view illustrating an example of the first member 61A of the ultraviolet light fluid treatment device 1A. FIG. 10 depicts a perspective view of the first member 61A and the first connection portion 18a located inside the ultraviolet light fluid treatment device 1A in a plan view. FIG. 11 is an enlarged view of a region L of FIG. 9. FIG. 12 is an enlarged view of a region M of FIG. 9.

As illustrated in FIG. 10, the first member 61A has three first openings 610A. Further, the first member 61A has third openings 612A leading to the second flow channel 82a and located under respective third portions 613A in regions where the third portions 613A are disposed. The first member 61A has six third openings 612A and six third portions 613A. The third portions 613A overlap the respective third openings 612A. Therefore, in FIG. 10, the reference numeral of a third opening 612A is written in parentheses and illustrated together with the reference numeral of a corresponding third portion 613A.

The shape of each of the first openings 610A and the third openings 612A in a plan view is a substantially rectangular shape with the longer side being in the direction orthogonal to the first direction d1 and to the second direction d2. The three first openings 610A and the six third openings 612A are arranged side by side in the first direction d1 over the substantially entire first member 61A. The area of each of the three first openings 610A is smaller than the area of the first connection portion 18a when the first member 61A is viewed in a plan view.

The number of the first openings 610A and the number of the third openings 612A are not particularly limited. The first member 61A may have at least one first opening 610A and at least one third opening 612A. Further, the arrangement of the at least one first opening 610A and the at least one third opening 612A in the first member 61A, the interval between adjacent first openings 610A, the interval between adjacent third openings 612A, and the like can be appropriately changed according to the use and the like of the ultraviolet light fluid treatment device 1A. The shape of each of the first openings 610A and the third openings 612A in a plan view is not limited to the substantially rectangular shape. The first openings 610A and the third openings 612A may have a substantially circular shape, a substantially square shape, a substantially elliptical shape, a substantially polygonal shape, or the like. Further, the plurality of first openings 610A may include first openings 610A having different shapes in a plan view. The plurality of third openings 612A may include third openings 612A having different shapes in a plan view.

First portions 611A are provided so as to overlap the respective first openings 610A in a plan view. In the example illustrated in FIG. 10, the first portions 611A are disposed under the respective three first openings 610A located on the downstream side in the first direction d1, and extend into the second flow channel 82a. The first portions 611A extend from regions adjacent to the first openings 610A into the second flow channel 82a. In the example illustrated in FIG. 11, a first portion 611A extends in the direction opposite to the second direction d2 from a region located downstream of a corresponding first opening 610A in the second direction d2. The cross-sectional shape of the first portion 611A orthogonal to the longitudinal direction of the first opening 610A is a curved shape protruding toward the second flow channel 82a such that the distance from the first opening 610A increases as the first portion 611A extends toward the direction opposite to the second direction d2.

The first portion 611A has a surface 614A that is inclined with respect to the second direction d2. The surface 614A is a surface having a curvature according to the shape of the first portion 611A. However, the cross-sectional shape of the first portion 611A orthogonal to the longitudinal direction of the first opening 610A is not limited to the curved shape, and may be any shape. Further, the surface 614A may be a surface having almost no curvature. The first portion 611A of the first member 61A can have a flat plate shape.

The third portions 613A are provided so as to overlap the respective third openings 612A in a plan view. In the example illustrated in FIG. 10, the third portions 613A are disposed above the respective six third openings 612A located on the upstream side in the first direction d1, and extend into the first flow channel 81a. The third portions 613A extend from regions adjacent to the third openings 612A into the first flow channel 81a. In the example illustrated in FIG. 12, a third portion 613A extends in the first direction d1 from a region located upstream of a corresponding third opening 612A in the first direction d1. The cross-sectional shape of the third portion 613A orthogonal to the longitudinal direction of the third opening 612A is a curved shape protruding toward the first flow channel 81a such that the distance from the third opening 612A increases as the third portion 613A extends toward the first direction d1.

The third portion 613A has a surface 615A that is inclined with respect to the first direction d1. The surface 615A is a surface having a curvature according to the shape of the third portion 613A. However, the cross-sectional shape of the third portion 613A orthogonal to the longitudinal direction of the third opening 612A is not limited to the curved shape, and may be any shape. Further, the surface 615A may be a surface having almost no curvature. The third portion 613A of the first member 61A can have a flat plate shape.

In the present embodiment, the first member 61A includes the first portion 611A. Therefore, the amount of the fluid flowing from the first flow channel 81a to the second flow channel 82a is larger than the amount of the fluid flowing from the second flow channel 82a to the first flow channel 81a. Accordingly, the flow of the fluid in the second flow channel 82a can be smoothed while reducing the influence on the flow from the first flow channel 81a to the second flow channel 82a in the connection portion. As a result, the time during which the fluid is irradiated with the ultraviolet light increases, and thus, the integrated illuminance can be increased, and treatment effects by the ultraviolet light fluid treatment device 1A can be enhanced.

Further, in the present embodiment, the first portion 611A has the surface 614A that is inclined with respect to the second direction d2. Therefore, a difference between the amount of the fluid flowing from the second flow channel 82a to the first flow channel 81a and the amount of the fluid flowing from the first flow channel 81a to the second flow channel 82a can be increased as compared to when the first portion 611A does not have the inclined surface 614 A. Accordingly, effects of smoothing the flow of the fluid in the second flow channel 82a can be increased. Thus, similar to the above, the integrated illuminance of the ultraviolet light emitted to the fluid can be increased, and treatment effects by the ultraviolet light fluid treatment device 1A can be enhanced.

Further, in the present embodiment, the first member 61A includes the third portion 613A. Therefore, the amount of the fluid flowing from the first flow channel 81a through the third opening 612A to the second flow channel 82a can be reduced. Accordingly, the integrated illuminance of the ultraviolet light emitted to the fluid flowing in the first flow channel 81a can be increased, and treatment effects by the ultraviolet light fluid treatment device 1A can be enhanced. Further, in the present embodiment, the first member 61A includes the third opening 612A. Therefore, the fluid can be easily discharged through the third opening 612A when the ultraviolet light fluid treatment device is emptied in order to prevent the growth of mold and bacteria. Accordingly, the growth of mold and bacteria in the ultraviolet light fluid treatment device due to residual fluid in the ultraviolet light fluid treatment device can be reduced. Thus, a decrease in treatment efficiency due to mold and bacteria can be reduced, and treatment effects by the ultraviolet light fluid treatment device 1A can be enhanced.

### (Second Member 62A)

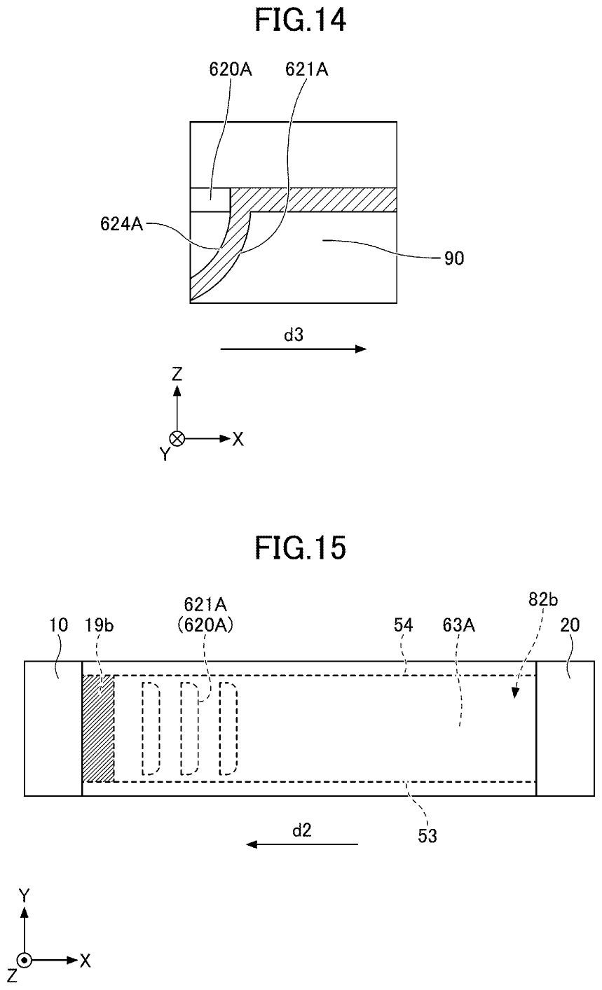

A detailed configuration of the second member 62A will be described with reference to FIG. 13 and FIG. 14. FIG. 13 is a schematic plan view illustrating an example of the second member 62A of the ultraviolet light fluid treatment device 1A. FIG. 13 depicts a perspective view of the second member 62A and second connection portion 19a located inside the ultraviolet light fluid treatment device 1A in a plan view. FIG. 14 is an enlarged view of a region N of FIG. 9.

As illustrated in FIG. 13, the second member 62A has three second openings 620A. The second openings 620A are holes penetrating the second member 62A in the thickness direction of the second member 62A. The shape of each of the second openings 620A in a plan view is a substantially rectangular shape with the longer side being in the direction orthogonal to the first direction d1 and to the second direction d2. The three second openings 620A are arranged side by side and provided downstream of a center M2A of the second member 62A in the second direction d2. The area of each of the three second openings 620A is smaller than the area of the second connection portion 19a when the second member 62A is viewed in a plan view.

The number of the second openings 620A is not particularly limited. The second member 62A may have at least one second opening 620A. Further, the arrangement of the at least one second opening 620A in the second member 62A, the interval between adjacent second openings 620A, and the like can be appropriately changed according to the use and the like of the ultraviolet light fluid treatment device 1A. The shape of the second openings 620A in a plan view is not limited to the substantially rectangular shape. The second openings 620A may have a substantially circular shape, a substantially square shape, a substantially elliptical shape, a substantially polygonal shape, or the like. Further, the plurality of second openings 620A may include second openings 620A having different shapes in a plan view.

Second portions 621A are provided so as to overlap the respective second openings 620A in a plan view. In the example illustrated in FIG. 13, the second portions 621A are disposed under the three respective second openings 620A located on the downstream side in the second direction d2, and extend into the third flow channel 90. The second portions 621A extend from regions adjacent to the seconds opening 620A into the third flow channel 90. In the example illustrated in FIG. 14, a second portion 621A extends into the third flow channel 90 from a region located downstream of a corresponding second opening 620A in the third direction d3. The cross-sectional shape of the second portion 621A orthogonal to the longitudinal direction of the second opening 620A is a half-arch shape or a curved shape protruding toward the upstream side in the third direction d3.

The second portion 621A has a surface 624A that is inclined with respect to the third direction d3. The surface 624A is a surface having a curvature according to the shape of the second portion 621A. However, the cross-sectional shape of the second portion 621A orthogonal to the longitudinal direction of the second opening 620A is not limited to the curved shape, and may be any shape. Further, the surface 624A may be a flat surface having almost no curvature. The second member 62A can have a louver structure in which flat-plate shaped second portions 621A are arranged.

In the present embodiment, the second member 62A includes the second portion 621A. Therefore, the amount of the fluid flowing from the second flow channel 82a to the third flow channel 90 is larger than the amount of the fluid flowing from the third flow channel 90 to the second flow channel 82a. Accordingly, the flow from the second flow channel 82a to the third flow channel 90 is stabilized, and thus, the flow of the fluid in the third flow channel 90 can be smoothed and the amount of the fluid flowing at a high velocity in the third flow channel 90 can be reduced. As a result, the time during which the fluid is irradiated with the ultraviolet light increases. Thus, the integrated illuminance can be increased, and treatment effects by the ultraviolet light fluid treatment device 1A can be enhanced.

Further, in the present embodiment, the, second portion 621A has the surface 624A that is inclined with respect to the third direction d3. Therefore, a difference between the amount of the fluid flowing from the third flow channel 90 to the second flow channel 82a and the amount of the fluid flowing from the second flow channel 82a to the third flow channel 90 can be increased as compared to when the second portion 621A does not have the inclined surface 624A. Accordingly, effects of smoothing the flow of the fluid in the third flow channel 90 can be increased. Thus, similar to the above, the integrated illuminance of the ultraviolet light emitted to the fluid can be increased, and treatment effects by the ultraviolet light fluid treatment device 1A can be enhanced.

### (Third Member 63A and Fourth Member 64A)

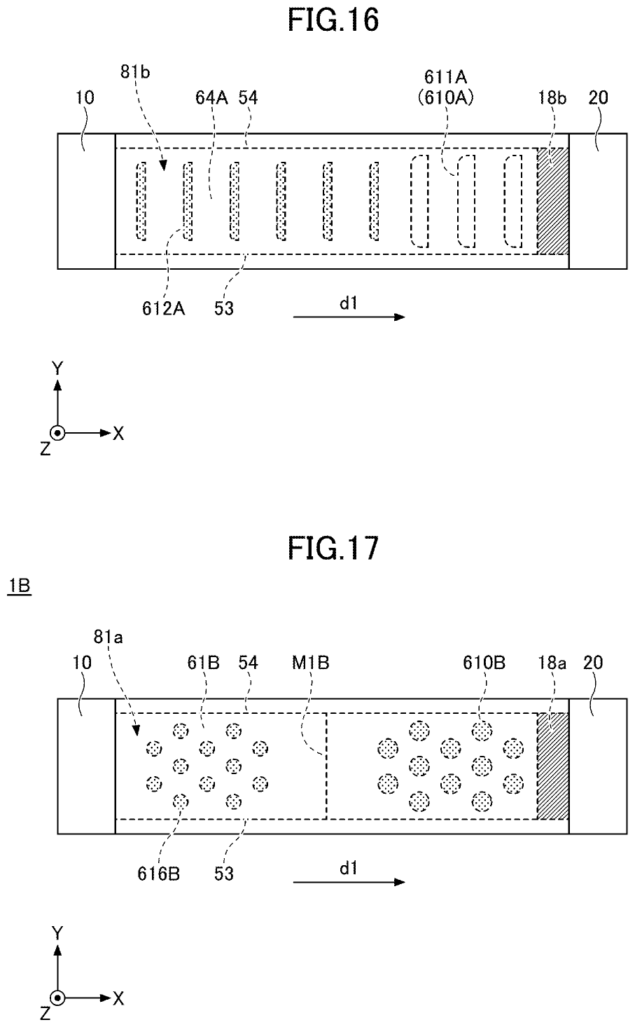

FIG. 15 is a schematic plan view illustrating an example of the third member 63A of the ultraviolet light fluid treatment device 1A. FIG. 15 depicts a perspective view of the third member 63A and the second connection portion 19b located inside the ultraviolet light fluid treatment device 1A in a plan view. FIG. 16 is a schematic plan view illustrating an example of the fourth member 64A of the ultraviolet light fluid treatment device 1A. FIG. 16 depicts a perspective view of the fourth member 64A and the first connection portion 18b located inside the ultraviolet light fluid treatment device 1A in a plan view.

The configuration and effects of the third member 63A are substantially the same as those of the second member 62A, and the configuration and effects of the fourth member 64A are substantially the same as those of the first member 61A. Thus, the description of the third member 63A and the fourth member 64A the same as those described above will not be repeated.

### Third Embodiment

Next, an ultraviolet light fluid treatment device according to a third embodiment will be described. The third embodiment differs from the above-described embodiments in that a first member further has a plurality of fourth openings connecting the first flow channel and the second flow channel, a plurality of first openings is provided only in a region downstream of the center of the first member in the first direction, and the plurality of fourth openings is provided only in a region upstream of the center of the first member in the first direction.

FIG. 17 is a schematic plan view illustrating a first member 61B of an ultraviolet light fluid treatment device 1B according to the third embodiment. The ultraviolet light fluid treatment device 1B has the same configuration as the ultraviolet light fluid treatment device 1 according to the first embodiment, except that the ultraviolet light fluid treatment device 1B includes the first member 61B instead of the first member 61. FIG. 17 depicts a perspective view of the first member 61B and the first connection portion 18a located inside the ultraviolet light fluid treatment device 1B in a plan view. The description will be given with reference to FIG. 1 and FIG. 2 as appropriate.

As illustrated in FIG. 17, the first member 61B of the ultraviolet light fluid treatment device 1B has twelve first openings 610B and twelve fourth openings 616B. The first openings 610B and the fourth openings 616B connect the first flow channel 81a and the second flow channel 82a. The first openings 610B and the fourth openings 616B are holes penetrating the first member 61B in the thickness direction of the first member 61B. The shape of each of the first openings 610B and the fourth openings 616B in a plan view is a substantially circular shape. However, the shape of the first openings 610B and the fourth openings 616B in a plan view may be a substantially rectangular shape, a substantially elliptical shape, a substantially polygonal shape, or the like. The substantially rectangular shape includes a substantially rectangular shape with the longer side being in the direction orthogonal to the first direction d1 in a plan view.

The twelve first openings 610B are provided only in a region downstream of a center M1B of the first member 61B in the first direction d1. Thus, no first opening 610B is provided in a region upstream of the center M1B of the first member 61B in the first direction d1. The twelve fourth openings 616B are provided only in the region upstream of the center M1B of the first member 61B in the first direction d1. Thus, no fourth opening 616B is provided in the region downstream of the center M1B of the first member 61B in the first direction d1.

The twelve first openings 610B have the same shape. Further, the twelve fourth openings 616B have the same shape. When the first member 61B is viewed from the first flow channel 81a side in a plan view, the area of each of the first openings 610B is larger than the area of each of the fourth openings 616B. Therefore, the sum of the areas of the twelve first openings 610B is greater than the sum of the areas of the twelve fourth openings 616B.

The number of the first openings 610B, the number of the fourth openings 616B, the interval between adjacent first openings 610B, the interval between adjacent fourth openings 616B, and the like can be appropriately changed according to the use and the like of the ultraviolet light fluid treatment device 1B. The arrangement of the first openings 610B can be changed as appropriate in the region downstream of the center M1B of the first member 61B in the first direction d1. The arrangement of the fourth openings 616B can be changed as appropriate in the region upstream of the center M1B of the first member 61B in the first direction d1.

According to the present embodiment, with the above configuration, the amount of the fluid flowing from the first flow channel 81a to the second flow channel 82a through the first openings 610B in the region downstream of the center M1B of the first member 61B in the first direction d1 is larger than the amount of the fluid taking a shortcut from the first flow channel 81a to the second flow channel 82a through the fourth openings 616B in the region upstream of the center M1B of the first member 61B in the first direction d1. As a result, the integrated luminance can be increased and the amount of fluid to be treated can be increased. Accordingly, treatment effects by the ultraviolet light fluid treatment device 1B can be improved.

### Other Embodiments

### First Example

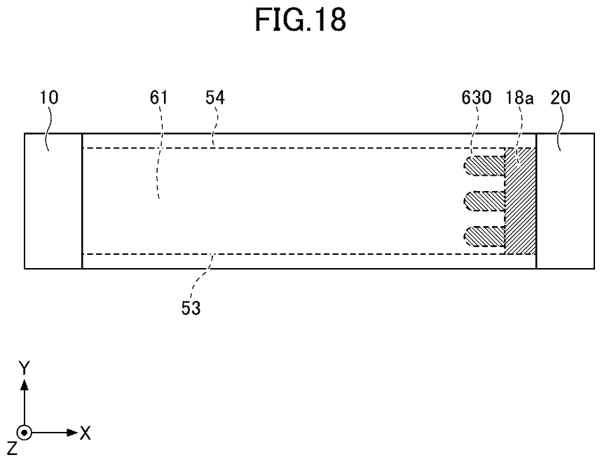

The ultraviolet light fluid treatment device according to any of the embodiments may have grooves in one or more of the first member 61, the second member 62, the third member 63, and the fourth member 64. FIG. 18 is a schematic plan view illustrating an example of a first member 61 having grooves. FIG. 18 depicts a perspective view of the first member 61 and the first connection portion 18a located inside the ultraviolet light fluid treatment device in a plan view. The first member 61 and the first connection portion 18a are located between the first end portion 10 and the second end portion 20 in a direction along the X-axis, and are located between the third wall 53 and the fourth wall 54 in a direction along the Y-axis.

As illustrated in FIG. 18, the first member 61 has three grooves 630 at the end, closer to the second end portion 20, of the first member 61. Each of the three grooves 630 has a substantially rectangular shape in a plan view. The three grooves 630 are arranged at substantially equal intervals in the direction along the Y-axis. In the ultraviolet light fluid treatment device according to any of the embodiments, the flow of the fluid in the ultraviolet light fluid treatment device can be easily smoothed by having the grooves 630 in the first member 61.

The number of the grooves 630 is not limited to three, and may be any number greater than or equal to one. The shape of the grooves 630 in a plan view is not limited to the substantially rectangular shape, and may be any shape. If the first member 61 has a plurality of grooves 630, the grooves 630 are not necessarily arranged at substantially equal intervals. In addition, the plurality of grooves 630 may have different shapes in a plan view.

The first member 61 may have first openings 610, first openings 610A, second openings 620, second openings 620A, or the like in addition to the grooves 630. The ultraviolet light fluid treatment device according to any of the embodiments may have the grooves 630 in one or more of the first member 61, the second member 62, the third member 63, and the fourth member 64.

### Second Example

The ultraviolet light fluid treatment device according to any of the embodiments may include one or both of the drain port 120 and the exhaust port 130 in either the third wall 53 or the fourth wall 54, such that the ultraviolet light fluid treatment device can be installed with the orientation of the ultraviolet light fluid treatment device being rotated by 90 degrees. The expression “when the ultraviolet light fluid treatment device is installed with the orientation of the ultraviolet light fluid treatment device being rotated by 90 degrees” refers to, for example, when the ultraviolet light fluid treatment device is installed with either the third wall 53 or the fourth wall 54 facing vertically downward.

### Third Example

The light source 170 illustrated in any of FIG. 3A through FIG. 3C may include jack mechanisms instead of the spring members 176, and may be configured such that the second surface 170b is pressed by the jack mechanisms against the first partition wall 13b that separates the upstream flow channel 12 and the first light source placement portion 13.

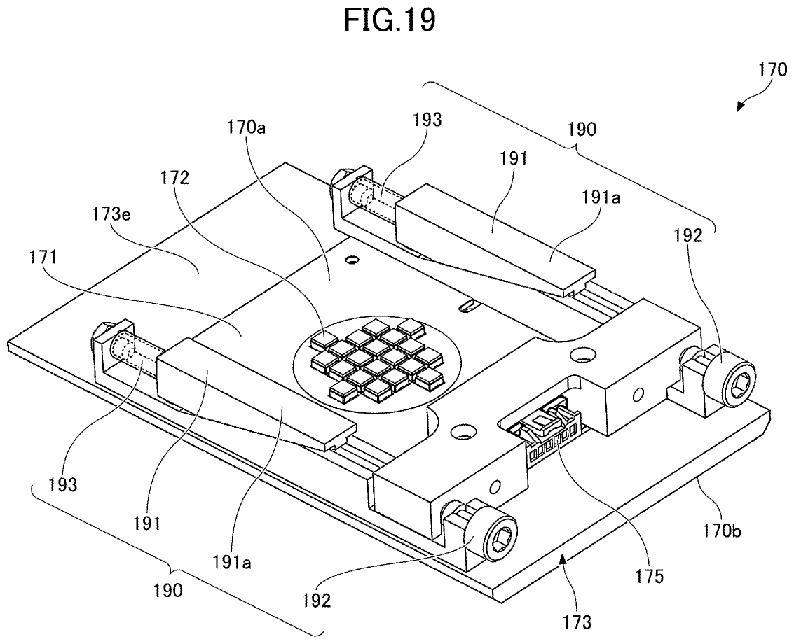

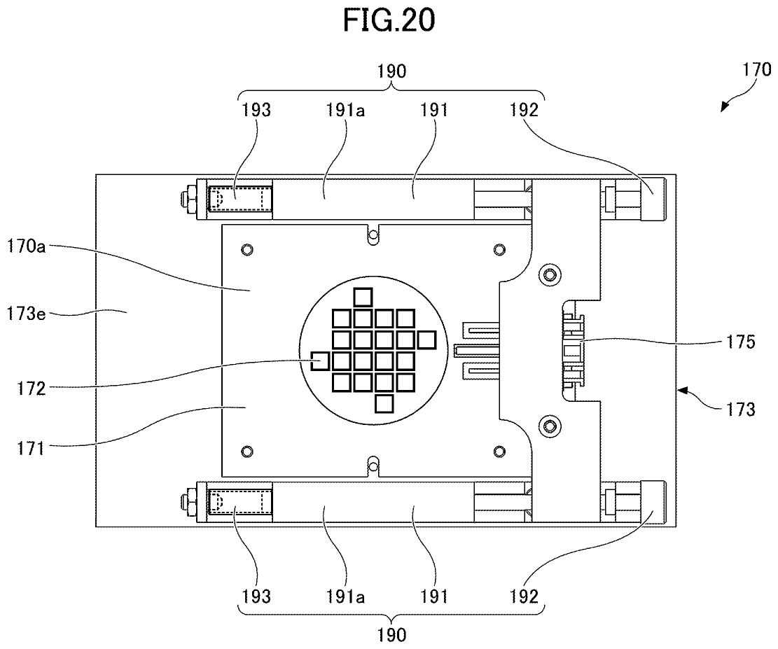

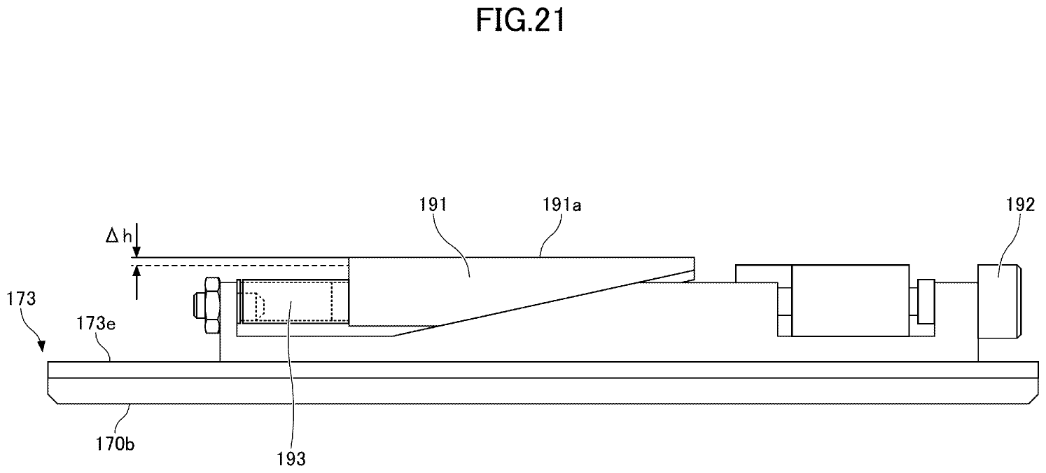

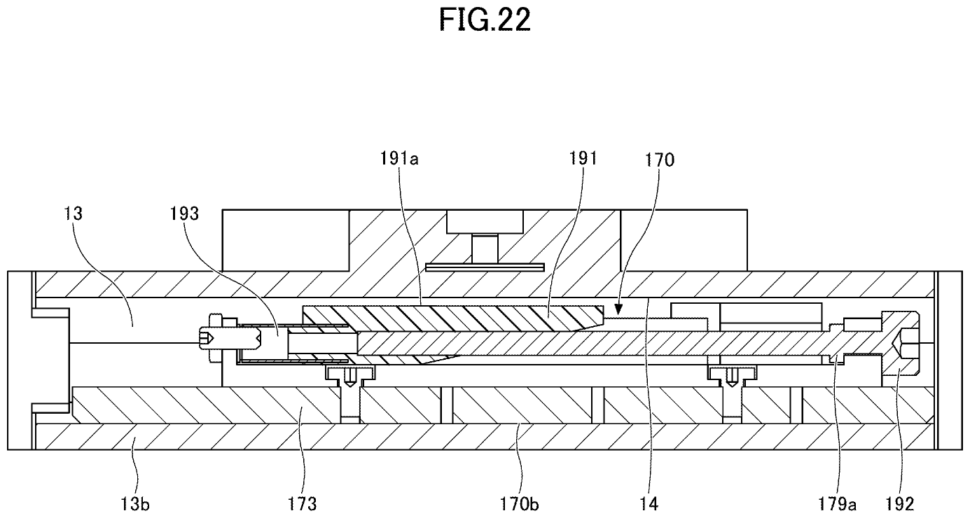

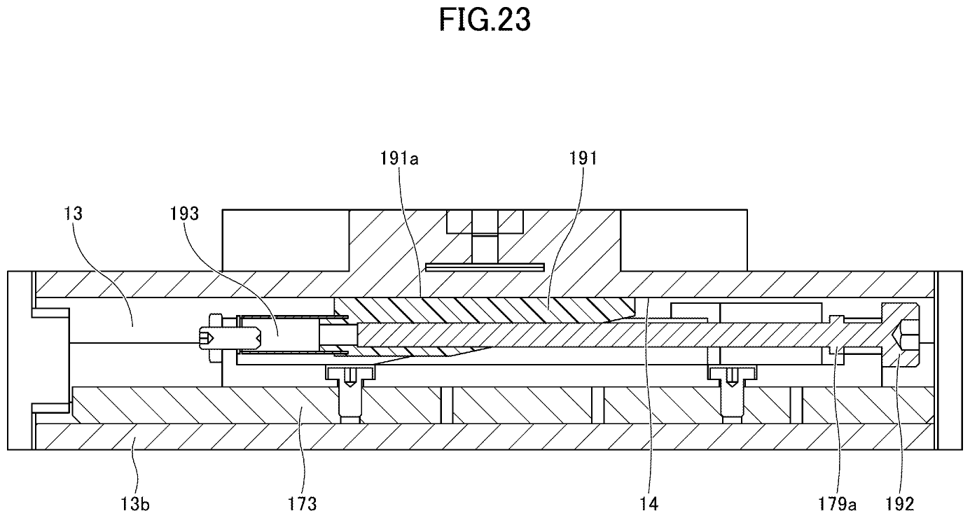

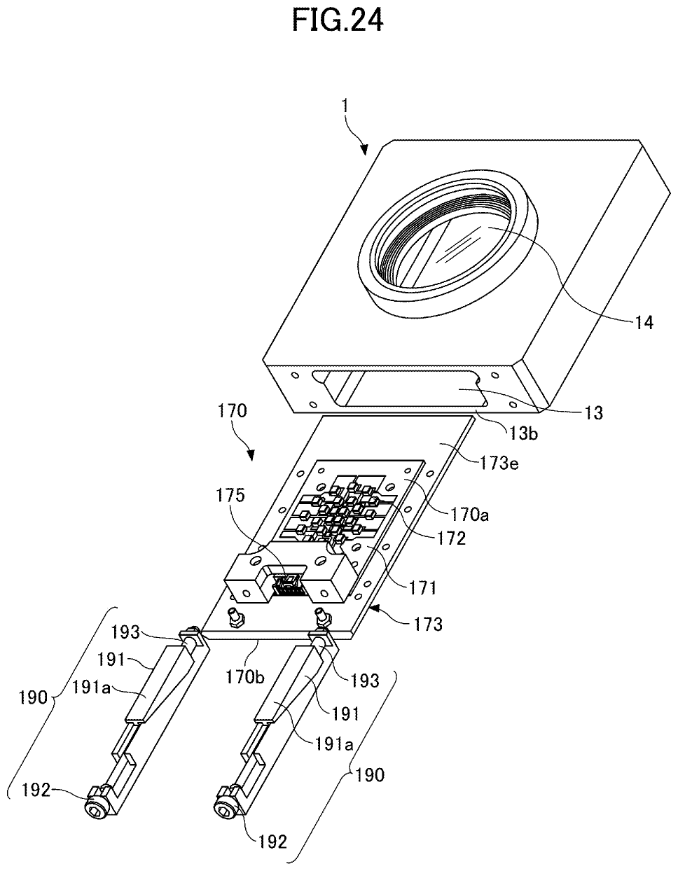

FIG. 19 through FIG. 23 are diagram illustrating jack mechanisms 190 of a light source 170. FIG. 19 is a perspective view, FIG. 20 is a top view, and FIG. 21 is a side view of the light source 170 including the jack mechanisms 190. FIG. 22 is a cross-sectional view illustrating a state before the light source 170 is pressed against the first partition wall 13b of the first light source placement portion 13. FIG. 23 is a cross-sectional illustrating a state in which the light source 170 is being pressed against the first partition wall 13b.

As illustrated in FIG. 19 and FIG. 20, the jack mechanisms 190 are disposed on the first surface 170a side of the light source 170. For example, a wiring substrate 171 is positioned between the two jack mechanisms 190 in a plan view and is fixed to a holding member 173. Each of the jack mechanisms 190 includes a support member 191 having a top surface 191a, a screw member 192, and an auxiliary spring member 193.

The support member 191 is composed of, for example, a resin material, a metal material, or the like. The support member 191 is coupled to the screw member 192. As illustrated in FIG. 21, the top surface 191a of the support member 191 can move in a direction normal to the top surface 191a by the rotation of the screw member 192. The amount of movement Δh illustrated in FIG. 21 indicates the amount of movement of the top surface 191a of the support member 191 by the rotation of the screw member 192. A dashed line in FIG. 21 indicates the position of the top surface 191a of the support member 191 in a state in which the top surface 191a is lowered (in a state in which the support member 191 is moved toward the second surface 170b). A solid line in FIG. 21 indicates the position of the top surface 191a of the support member 191 in a state in which the top surface 191a is raised (in a state in which the support member 191 is moved to the side opposite to the second surface 170b). The amount of movement Δh is, for example, 1 mm. The auxiliary spring member 193 applies a force that preloads the support member 191 toward the screw head of the screw member 192.

As illustrated in FIG. 22, the light source 170 can be disposed in the first light source placement portion 13 in a state in which the top surface 191a is lowered in the direction normal to the top surface 191a, that is, in a state in which there is a gap between the top surface 191a and the first window 14 facing the top surface 191a. In the present embodiment, the light source 170 is disposed in the first light source placement portion 13 in a state in which the top surface 191a is lowered. In this manner, the light source 170 can be easily disposed. Upon the screw member 192 being rotated by an operator after the light source 170 is disposed in the first light source placement portion 13, the top surface 191a moves toward the first window 14 and the top surface 191a contacts the first window 14. Upon the screw member 192 being further rotated with the top surface 191a contacting the first window 14, the second surface 170b is pressed against the first partition wall 13b. With this configuration, according to the present embodiment, the cooling efficiency of the light source 170 by the fluid flowing in the upstream flow channel 12 can be enhanced.

The light source 170 may be disposed in each of the second light source placement portions 16. The light source 170 can be disposed in each of the second light source placement portions 16 in a state in which there is a gap between the top surface 191a and the second window 17 facing the top surface 191a. In the present embodiment, the light source 170 is disposed in each of the second light source placement portions 16 in a state in which the top surface 191a is lowered. In this manner, the light source 170 can be easily disposed. Upon the screw member 192 being rotated by an operator after the light source 170 is disposed in each of the second light source placement portions 16, the top surface 191a moves toward the second window 17 and the top surface 191a contacts the second window 17. Upon the screw member 192 being further rotated with the top surface 191a contacting the second window 17, the second surface 170b is pressed against the second partition wall 16b. With this configuration, according to the present embodiment, the cooling efficiency of the light source 170 by the fluid flowing in the downstream flow channel 110 can be enhanced.

As illustrated in FIG. 19 and FIG. 20, in this example, a plurality of housings 172 including light-emitting elements is arranged, such that one or more housings 172 of the plurality of housings 172 are arranged in a substantially rectangular shape as viewed in a direction in which light is emitted from the light emitting elements, and one housing 172 of the plurality of housings 172 is disposed on each of the outer sides of the rectangle. Two or more housings 172 may be disposed on each of the outer sides of the rectangle. In the ultraviolet light fluid treatment device according to any of the embodiments, if the cross-sectional shapes of the flow channels in a direction orthogonal to the directions in which the fluid flows is a substantially circular shape, the plurality of housings 172 including the light-emitting elements is preferably arranged as illustrated in FIG. 19 and FIG. 20. The ultraviolet light fluid treatment device according to any of the embodiments can efficiently emit the light from the light-emitting elements to the fluid in the flow channels.

However, the arrangement of the plurality of housings 172 can be appropriately changed according to the shape of the flow channels and the like. For example, if the cross-sectional shape of the flow channels is a substantially circular shape, the plurality of housings 172 may be arranged concentrically as viewed in the direction in which the light is emitted from the light-emitting elements.

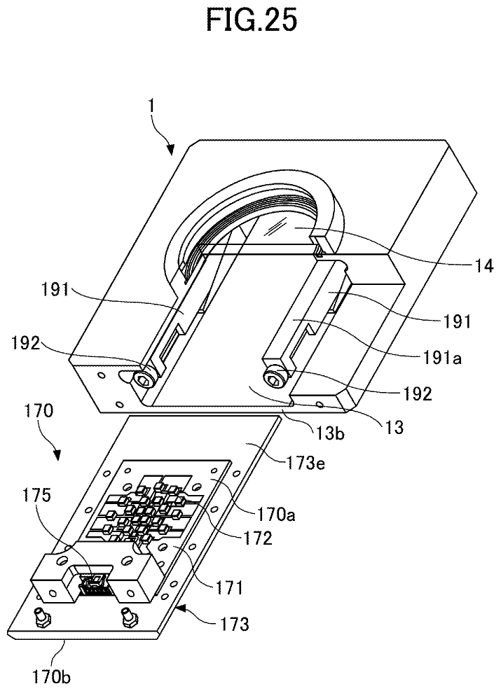

FIG. 24 and FIG. 25 are exploded perspective views illustrating a method of disposing the jack mechanisms 190 in the ultraviolet light fluid treatment device 1 according to the embodiment. FIG. 25 depicts a partial cross-sectional view of the first light source placement portion 13 and the first window 14. FIG. 24 depicts a first example of a method of disposing the jack mechanisms 190, and FIG. 25 depicts a second example of a method of disposing the jack mechanisms 190. In FIG. 24 and FIG. 25, a configuration in the vicinity of the first window 14 of the ultraviolet light fluid treatment device 1 is extracted and depicted. In the examples illustrated in FIG. 24 and FIG. 25, the jack mechanisms 190 are disposed in the ultraviolet light fluid treatment device 1 as separate members from the light source 170. That is, the jack mechanisms 190 are not integrated with the light source 170.

In the example illustrated in FIG. 24, first, the light source 170 is inserted into the first light source placement portion 13 of the ultraviolet light fluid treatment device 1. Then, the jack mechanisms 190 are inserted into the first light source placement portion 13. The jack mechanisms 190 are inserted into the first light source placement portion 13 in a state in which top surfaces 191a are lowered. After the jack mechanisms 190 are inserted into the first light source placement portion 13, the second surface 170b is pressed against the first partition wall 13b by rotating screw members 192. Conversely, in the example illustrated in FIG. 25, the jack mechanisms 190 are fixed to the first light source placement portion 13. The light source 170 is inserted between the first partition wall 13b and the jack mechanisms 190 fixed to the first light source placement portion 13. The jack mechanisms 190 are not necessarily inserted into the first light source placement portion 13 through the first light source opening 13a provided in the lateral surface 10a illustrated in FIG. 1, and may be inserted into the first light source placement portion 13 through an opening provided in a surface opposite to the lateral surface 10a. Further, the top surfaces 191a of the jack mechanisms 190 do not necessarily contact the first window 14, and the top surfaces 191a may contact a surface opposite to the first window 14 in the first light source placement portion 13. The surface opposite to the first window 14 in the first light source placement portion 13 is the surface 173e of the holding member 173, the first surface 170a of the light source 170, or the like.

Although specific embodiments have been described above, the present disclosure is not limited to the above-described embodiments. The above-described embodiments may be modified by a person skilled in the art as long as the features of the present disclosure are included. In addition, various changes and modifications can be conceived by a person skilled in the art within the spirit and scope of the present disclosure, and it is understood that the changes and modifications are included in the spirit and scope of the present disclosure.

For example, in each of the above-described embodiments, the fluid entering the third flow channel 90 flows into each of the outlet 15 and the downstream flow channel 110; however, all of the fluid entering the third flow channel 90 may flow to the downstream flow channel 110. For example, if the fluid entering the third flow channel 90 flows into each of the outlet 15 and the downstream flow channel 110, there may be a case where the fluid does not reach the vicinity of each of the upper and lower ends in the vertical direction of the downstream flow channel 110. In the regions where the fluid does not reach, cooling effects of the second light sources 72 may be insufficient. By allowing all of the fluid entering the third flow channel 90 to flow into the downstream flow channel 110, the fluid can reach the vicinity of each of the upper and lower ends in the vertical direction of the downstream flow channel 110. Accordingly, cooling effects of the second light sources 72 can be appropriately obtained.

According to an embodiment of the present disclosure, an ultraviolet light fluid treatment device that can enhance treatment effects can be provided.

What is claimed is:

1. An ultraviolet light fluid treatment device comprising:
a first flow channel in which fluid flows in a first direction;
a second flow channel that is connected to a downstream side of the first flow channel and in which the fluid flows in a second direction opposite to the first direction;
a first member disposed between the first flow channel and the second flow channel;
a light source configured to emit ultraviolet light to one or both of the first flow channel and the second flow channel; and
a first connection portion connecting a downstream end of the first flow channel and an upstream end of the second flow channel,
wherein the first member has a first opening connecting the first flow channel and the second flow channel, and
an area of the first opening is smaller than an area of the first connection portion in a plan view.

2. The ultraviolet light fluid treatment device according to claim 1, wherein the first opening is provided in a region downstream of a center of the first member in the first direction.

3. The ultraviolet light fluid treatment device according to claim 1, wherein the first member includes a first portion extending from a region adjacent to the first opening into the second flow channel.

4. The ultraviolet light fluid treatment device according to claim 3, wherein the first portion has a surface that is inclined with respect to the second direction.

5. The ultraviolet light fluid treatment device according to claim 1, further comprising:
a third flow channel that is connected to a downstream side of the second flow channel and in which the fluid flows in a third direction opposite to the second direction;
a second member disposed between the second flow channel and the third flow channel; and
a second connection portion connecting a downstream end of the second flow channel and an upstream end of the third flow channel,
wherein the second member has a second opening connecting the second flow channel and the third flow channel, and
an area of the second opening is smaller than an area of the second connection portion in the plan view.

6. The ultraviolet light fluid treatment device according to claim 5, wherein the second opening is provided in a region downstream of a center of the second member in the second direction.

7. The ultraviolet light fluid treatment device according to claim 5, wherein the second member includes a second portion extending from a region adjacent to the second opening into the third flow channel.

8. The ultraviolet light fluid treatment device according to claim 7, wherein the second portion has a surface that is inclined with respect to the third direction.

9. The ultraviolet light fluid treatment device according to claim 7, wherein the second opening is provided at a position that does not overlap the first opening in the plan view.

10. The ultraviolet light fluid treatment device according to claim 1, wherein the first member includes a third opening and a third portion,
the third opening is provided upstream of the first opening of the first member in the first direction, and connects the first flow channel and the second flow channel, and
the third portion extends from a region adjacent to the third opening into the first flow channel.

11. The ultraviolet light fluid treatment device according to claim 10, wherein the third portion has a surface that is inclined with respect to the first direction.

12. The ultraviolet light fluid treatment device according to claim 1, wherein the first member further has a plurality of fourth openings connecting the first flow channel and the second flow channel,
the first opening includes a plurality of first openings provided in a region downstream of a center of the first member in the first direction,
the plurality of fourth openings is provided in a region upstream of the center of the first member in the first direction, and
a sum of areas of the plurality of first openings is greater than a sum of areas of the plurality of fourth openings in the plan view.

13. The ultraviolet light fluid treatment device according to claim 1, further comprising:
an inlet of the fluid;
an outlet of the fluid; and
a plurality of branch flow channels branching from the inlet, and
a merged flow channel connected to a downstream side of each of the branch flow channels, and
each of the branch flow channels includes the first flow channel and the second flow channel between which the first member is disposed.
